(19) United States
(12) Patent Application Publication
(10) Pub. No.: US 2006/0045865 A1
(43) Pub. Date: Mar. 2, 2006

(54) Controlled regional oral delivery

(75) Inventors: Jules S. Jacob, Taunton, MA (US); Edith Mathiowitz, Brookline, MA (US); Avinash Nangia, Wrentham, MA (US); Ze'ev Shaked, San Antonio, TX (US); Peyman Moslemy, Providence, RI (US)

Correspondence Address: PATREA L. PABST;PABST PATENT GROUP LLP, 400 COLONY SQUARE, SUITE 1200, ATLANTA, GA, 30361, US

(73) Assignee: Spherics, Inc.

(21) Appl. No.: 11/214,206

(22) Filed: Aug. 28, 2005

Related U.S. Application Data
(60) Provisional application No. 60/604,990, filed on Aug. 27, 2004.
(60) Provisional application No. 60/605,198, filed on Aug. 27, 2004.
(60) Provisional application No. 60/605,199, filed on Aug. 27, 2004.
(60) Provisional application No. 60/605,200, filed on Aug. 27, 2004.
(60) Provisional application No. 60/605,201, filed on Aug. 27, 2004.
(60) Provisional application No. 60/607,905, filed on Sep. 8, 2004.
(60) Provisional application No. 60/650,191, filed on Feb. 4, 2005.
(60) Provisional application No. 60/650,375, filed on Feb. 4, 2005.

(51) Int. Cl. A61K 31/74

(52) U.S. Cl. 424/78.27

(57) ABSTRACT

A composite formulation has been developed for selective, high efficacy delivery to specific regions of the mouth and gastrointestinal tract. The formulation is typically in the form of a tablet or capsule, which may include microparticles or beads. The formulation uses bioadhesive and controlled release elements to direct release to specific regions, where the drug is absorbed in enhanced amounts relative to the formulation in the absence of the bioadhesive and/or controlled release elements. This is demonstrated by an example showing delivery of gabapentin with a greater area under the curve (“AUC”) relative to the FDA reference immediate release drug, i.e., the AUC of the composite bioadhesive formulation is greater than 100% of the AUC of the immediate release drug. In the preferred embodiments, the formulation includes drug to be delivered, controlled release elements, and one or more bioadhesive elements. The bioadhesive polymer may be either dispersed in the matrix of the tablet or applied as a direct compressed coating to the solid oral dosage form. The controlled release elements are selected to determine the site of release. The bioadhesive components are selected to provide retention of the formulation at the desired site of uptake and administration. By selecting for both release and retention at a specific site, typically based on time of transit through the gastrointestinal tract, one obtains enhanced efficacy of uptake of the drug. This is particularly useful for drugs with narrow windows of absorption, and drugs with poor solubility such as the BCE class III and class IV drugs.

### CROSS-REFERENCE TO RELATED APPLICATIONS

This application claims priority to U.S. Ser. No. 60/605,199 filed on Aug. 27, 2004 “Mucoadhesive Oral Formulations of Low Permeability, Low Solubility Drugs”; U.S. Ser. No. 60/604,990 filed Aug. 27, 2004, entitled “Bioadhesive Rate Controlled Oral Dosage Formulation”; U.S. Ser. No. 60/607,905, filed Sep. 8, 2004 entitled “Mucoadhesive Oral Formulations Of High Permeability, High Solubility Drugs”; U.S. Ser. No. 60/650,191 filed Feb. 4, 2005, entitled “Bioadhesive Oral Formulations of High Permeability, High Solubility Drugs”; U.S. Ser. No. 60/605,201 filed Aug. 27, 2004 and U.S. Ser. No. 60/650,375 filed Feb. 4, 2005 entitled “Mucoadhesive Oral Formulations of High Permeabilty; U.S. Ser. No. 60/605,200 filed on Aug. 27, 2004 “Mucoadhesive Ulcer Formulation”; U.S. Ser. No. 60/605,198 filed Aug. 27, 2004 entitled “Multi-layer Dosage Form for Controlled Release of Active Substances”.

### FIELD OF THE INVENTION

The present invention is generally in the field of oral drug delivery and is in particular a method and compositions for oral delivery of drugs to precise regions of the gastrointestinal tract where there is a high degree of uptake.

### BACKGROUND OF THE INVENTION

The first oral drug delivery occurred thousands of years ago when individuals chewed herbs or drank extracts of compounds having a beneficial effect. This rather crude approach was greatly enhanced with the administration of purified compound, again either in the form of a solid, which was swallowed or in a liquid suspension. The art of compounding was developed as a means of insuring that accurate dosages where administered, then later as a means of improving patient compliance through incorporation of flavoring and coloring agents.

Even though many types of drugs could be administered orally with great efficacy, some classes of drugs remained a problem, especially those which were destroyed upon passage through the highly acidic conditions in the stomach. Accordingly, the next great improvement in drug delivery was the development of the enteric coating, a polymer coating applied to the outside of the drug formulation to protect the drug as it passed through the stomach, providing delayed release in the small intestine where the pH increased towards 6.8 to 7. The advantage of these materials was not only that the drug was protected. but that the materials were not bioactives, and could be used without special regulatory approval for any number of drugs

Even though means had now been developed to delay release, release was still occurring immediately following dissolution of the enteric coating. Controlled or sustained release formulations, primarily in the form of beads or microparticles were then developed, which provided for sustained delivery of drug following dissolution of the outer capsule. The controlled or sustained release was again obtained through the use of polymeric materials and other excipients, generally applicable to a variety of drugs. Many variations of the materials and formulations have been developed to further manipulate time and place of release.

Despite progress in the field of controlling release, uptake of the drug was still primarily a function of diffusion through the gastrointestinal tract, where it was affected by the presence of food and dissolution of the drug per se. Numerous means for targeting uptake of drugs to the gastrointestinal wall were attempted in an effort to increase the efficacy of uptake. Examples included targeting through the use of antibodies and other ligands. The disadvantage of these materials, however, was that the materials were themselves considered to be bioactive ingredients, requiring additional regulatory approval, and expensive. An alternative was through the use of microparticle size, based on studies at Southern Research Institute by Tice, et al., U.S. Pat. No. 5,811,128, that selection of microparticle size under 5-10 microns resulted in uptake directly into the Peyer's patches. The microparticles did not increase efficacy of uptake, however.

In the early 1990's, Mathiowitz et al. described several classes of polymers which could be manipulated as bioadhesive coatings on oral drug formulations to increase efficacy of uptake of oral drugs through the mucosa. See U.S. Pat. No. 6,197,346, for example. Subsequent work has focused on the development of other excipients enhancing bioadhesion.

While each new development has improved oral delivery of drugs, increased efficacy of delivery is still needed.

It is therefore an object of the present invention to provide oral drug formulations with increased selectivity and efficacy of delivery.

It is a further object of the present invention to provide oral drug formulations with increased selectivity and efficacy of delivery to the buccal/sublingual sites, the stomach, the small intestine, or the colon.

It is another object of the present invention to provide oral drug formulations with increased bioavailability of the bioadhesive formulation as compared to a system with no adhesive component.

It is another objective of the present invention to maintain effective plasma concentration over extended duration after oral administration.

It is another objective of the present invention to reduce the Cmax often related with side/adverse effects.

It is another objective of the present invention to reduce the inter-subject variability inherent to many drugs with narrow absorption window.

It is another objective of the present invention to deliver topically the drug at its target site of action for longer duration with low systemic absorption. This is particularly important for drugs delivered in the colon to treat a variety of inflammatory bowl diseases.

### SUMMARY OF THE INVENTION

A composite formulation has been developed for selective, high efficacy delivery to specific regions of the gastrointestinal tract. The formulation is typically in the form of a tablet or capsule, which may include microparticles or beads. The formulation uses bioadhesive and controlled release elements to direct release to specific regions where bioadhesive elements are exposed at the time the formulation reaches the region of desired release. This can result in enhanced amounts relative to the formulation in the absence of the bioadhesive and/or controlled release elements. This is demonstrated by several example showing delivery of different drugs greater area under the curve (“AUC”) relative to the reference immediate release dosage form, i.e., the AUC of the composite bioadhesive formulation is greater than 100% of the AUC of the immediate release drug and/or the drug in a formulation of only the controlled release or bioadhesive elements. In the preferred embodiment, the area under the curve is at least 10%, 15%, 20%, 30%, 40%, 50%, 100%, or 200% of the reference formulation. The AUC is also typically greater than the AUC of non-bioadhesive controlled release formulations and bioadhesive/controlled release formulations where the release of drug is not coordinated with the exposure of the bioadhesive of the drug formulation.

In the preferred embodiments, the formulation includes drug to be delivered, controlled release elements, and one or more bioadhesive elements. The bioadhesive polymer may be either dispersed in the matrix of the solid oral dosage form or applied as a direct compressed coating to the solid oral dosage form. Preferred bioadhesive polymers include poly(adipic)anhydride “p(AA)” and poly(fumaric-co-sebacic)anhydride “p[FA:SA]”. Other preferred bioadhesive polymers include non-erodable polymers such as DOPA-maleic anhydride co polymer; isopthalic anhydride polymer; DOPA-methacrylate polymers; and DOPA-cellulosic based polymers. The controlled release elements are selected to determine the site of release. For example, an enteric coating can be used to delay release until the formulation reaches the ileum; additional controlled release elements can be used to further delay release so that release occurs within the first one-third of the small intestine; the second one-third; or the last one-third of the small intestine. The controlled release elements can also be selected to delay release until the drug formulation reaches the colon. The bioadhesive components are selected to provide retention of the formulation at the desired site of uptake. This will occur after the enteric coating, if present, dissolves, or immediately after administration if no coating is applied. By selecting for both release and retention at a specific site, typically based on time of transit through the gastrointestinal tract, one obtains enhanced efficacy of uptake of the drug. This is particularly useful for drugs with narrow windows of absorption, and drugs with poor solubility such as the BCE class III and class IV drugs.

In addition the performance of many BCS class I and II drugs vary among subjects based on their GI transit and more particularly gastric emptying under fed conditions. This becomes more critical for drugs with narrow absorption window and results in large inter-subject variability. By selecting for both release and retention at the specific site, especially within the gastric region, the inter-subject variability is reduced significantly.

### BRIEF DESCRIPTION OF THE DRAWINGS

FIG. 1A is a schematic of a solid oral dosage form of a multiparticulate formulation containing drug(s), excipients, and optionally permeation and or dissolution enhancers, encapsulated in a single hard gelatin or cellulose-based capsule or monolithic tablet. FIG. 1B is a schematic of a solid oral dosage form including a multiparticulate formulation, containing drug(s), excipients, a bioadhesive polymer composition, and optionally permeation and or dissolution enhancers, in a single hard gelatin or cellulose-based capsule, oe monolithic tablet, optionally coated with one or more layers of release rate controlling polymers or enteric polymers. FIG. 1C is a longitudinal section of a longitudinally compressed tablet (“LCT”) containing drugs and excipients, and optionally permeation and/or dissolution enhancers, disposed in two or three monolithic layers with a slow dissolving or insoluble plug at one end. The LCTs were coated peripherally with a single layer of impermeable PCL film that was heat-sealed to the tablet core. Optionally, bioadhesive Spheromer™ polymer layers comprising either Spheromer™ I (anhydride polymers), Spheromer™ II (anhydride oligomers blended with pharmaceutical polymers), Spheromer™ III (catechol-grafted anhydride polymers), or combinations of these polymers can also be applied over the impermeable coating without affecting drug release.

FIG. 2 is a graph of the Area under the curve (concentration in micrograms/ml over time in hours) for gabapentin (Neurontin™) compared with a bioadhesive, controlled release gabapentin formulation.

FIG. 3A is a graph of dissolution over time (hours) of sulfasalazine (Azulfidine-EN®) 500 mg tablets and bioadhesive polymer coated multiparticulate beads in gelatin capsules. FIG. 3B is a graph of plasma concentration over time (hours) of sulfasalazine measured using LC/MS/MS, comparing Azulfidine-EN® tablets, 500 mg and Spheromer™ III-coated multiparticulate in capsule formulations (Batch 1 and 2), from different formulations evaluated in cohorts of six beagle dogs in the fed state.

FIG. 4A is a graph of the mean concentration of Itraconazole in plasma in fed volunteers against time following a single 100 mg dose of Treatment A (Spherazole™ CR Type A, Spherics Inc. USA), Treatment B (Spherazole™ CR Type B, Spherics Inc. USA) or Treatment C (Sporanox® 100 mg, Janssen Pharmaceutica Products L.P. USA), n=8. FIG. 4B is a graph of the area under the plasma itraconazole versus time curve (AUC), maximum concentration (Cmax), time to maximum concentration (Tmax) were calculated and are indicated in the figure.

FIG. 5 is a graph which shows plasma acyclovir profiles of Zovirax® (Acyclovir) 400 mg, Immediate Release (IR) tablets were compared with tablets prepared with 400 mg acyclovir in a controlled release (CR) formulation, BioVir™, and 400 mg acyclovir, 300 mg in a controlled release formulation and 100 mg in an immediate release formulation (CR+) dosed to fed beagle dogs.

FIG. 6 is a graph which shows plasma valacyclovir (micrograms/ml) levels over time (hours) for a 500 mg Valtrex® valacyclovir formulation as compared to a 400 mg CR+ 100 mg IR valacyclovir formulation dosed to fed beagle dogs.

FIGS. 7A-D is a comparison of the plasma concentrations (ng/ml) of levodopa and carbidopa from Sinemet® CR tablets (FIG. 7A), bioadhesive trilayer tablets (FIG. 7B), bioadhesive trilayer tablets with drug inserts (FIG. 7C), and Levodopa-Carbidopa pellets prepared by low shear granulation followed by extrusion-spheronization, one formulation used without coating, and one formulation with a a first layer of Eudragit® RL 100 and with a second outer layer of Spheromer™ III polymer (FIG. 7D). orally administered to fed beagle dogs.

### DETAILED DESCRIPTION OF THE INVENTION

The GastroIntestinal Tract

In a normal human adult male, the GI tract is approximately 25 feet long and consists of the following components: 1) mouth (buccal cavity; includes salivary glands, mucosa, teeth and tongue); 2) pharynx; 3) esophagus and cardia; 4) stomach, which includes the antrum and pylorus; 5) intestine, including the small intestine, which has three parts—duodenum, jejunum, and ileum, and the large intestine, which also has three parts—cecum, colon (ascending colon, transverse colon, descending colon and sigmoid flexure) and rectum ; and 6) the anus. Under normal circumstances, a drug may be expected to remain in the stomach for 2 to 4 hours (gastric emptying time) and in the small intestine for 4 to 10 hours, although there is a substantial variation between people, and even in the same person on different occasions. The gastric emptying time for a dosage form is most rapid with a fasting stomach, becoming slower as the food content is increased. Changes in gastric emptying time and/or intestinal motility can affect dosage form transit time and thus the opportunity for drug dissolution and absorption. (Ansel et al. Pharmaceutical Dosage Forms and Drug Delivery Systems 6th ed. Williams and Wilkins, 1995). Generally drugs are better absorbed in the small intestine (because of the larger surface area) than in the stomach, therefore quicker stomach emptying will increase drug absorption. For example, a good correlation has been found between stomach emptying time and peak plasma concentration for acetaminophen. The quicker the stomach emptying (shorter stomach emptying time) the higher the plasma concentration. Also slower stomach emptying can cause increased degradation of drugs in the stomach's lower pH; e.g. proton pump inhibitors, carbidopa. Food can effect the rate of gastric emptying. For example fatty food can slow gastric emptying and retard drug absorption. Generally the extent of absorption is not greatly reduced. Occasionally absorption may be improved. Griseofulvin absorption is improved by the presence of fatty food. Apparently the poorly soluble griseofulvin is dissolved in the fat and then more readily absorbed.

The various gastrointestinal regions and typical transit times are shown in the following Table 1.

As described below, it is typically a coordinated combination of controlled release and bioadhesive elements that are used to achieve release in the desired region where enhanced uptake occurs due to the inclusion of the bioadhesive elements.

TABLE 1
Characteristics of Gastro-intestinal Physiology

| | | | Blood | Surface | Transit | Bypass |
|---|---|---|---|---|---|---|
| REGION | pH | Membrane | Supply | Area | Time | liver |
| BUCCAL | approx 6 | thin | Good, fast | small | Short | yes |
| | | | absorption | | unless | |
| | | | with low | | controlled | |
| | | | dose | | | |
| ESOPHAGUS | 6 | Very | — | small | short | — |
| | | thick, no | | | | |
| | | absorption | | | | |
| STOMACH | 1-3 | normal | good | small | 30-40 | no |
| | decomposition, | | | | minutes, | |
| | weak acid | | | | reduced | |
| | unionized | | | | absorption | |
| DUODENUM | 4-5.5 | normal | good | very | very short | no |
| | bile duct, | | | large | (6″ long), | |
| | surfactant | | | | window | |
| | properties | | | | effect | |
| SMALL | 6-7 | normal | good | very | about 3 | no |
| INTESTINE | | | | large | hours | |
| | | | | 10-14 ft, | | |
| | | | | 80 cm²/cm | | |
| LARGE | 6.8-7 | — | good | not | long, up | lower |
| INTESTINE | | | | very | to 24 hr | colon, |
| | | | | large 4-5 ft | | rectum |
| | | | | | | yes |

I. Formulations

A. Bioactive Agents

The Biopharmaceutical Classification System (BCS), originally developed by G. Amidon, separates pharmaceuticals for oral administration into four classes depending on their aqueous solubility and their permeability through the intestinal cell layer. According to the BCS, drug substances are classified as follows:

Class I—High Permeability, High Solubility

Class II—High Permeability, Low Solubility

Class III—Low Permeability, High Solubility

Class IV—Low Permeability, Low Solubility

The interest in this classification system stems largely from its application in early drug development and then in the management of product change through its life-cycle. In the early stages of drug development, knowledge of the class of a particular drug is an important factor influencing the decision to continue or stop its development. Class 1 drugs of the BCS system are highly soluble and highly permeable in the gastrointestinal (GI) tract.

The solubility class boundary is based on the highest dose strength of an immediate release (“IR”) formulation and a pH-solubility profile of the test drug in aqueous media with a pH range of 1 to 7.5. Solubility can be measured by the shake-flask or titration method or analysis by a validated stability-indicating assay. A drug substance is considered highly soluble when the highest dose strength is soluble in 250 ml or less of aqueous media over the pH range of 1-7.5. The volume estimate of 250 ml is derived from typical bioequivalence (BE) study protocols that prescribe administration of a drug product to fasting human volunteers with a glass (about 8 ounces) of water. The permeability class boundary is based, directly, on measurements of the rate of mass transfer across human intestinal membrane, and, indirectly, on the extent of absorption (fraction of dose absorbed, not systemic bioavailability) of a drug substance in humans. The extent of absorption in humans is measured using mass-balance pharmacokinetic studies; absolute bioavailability studies; intestinal permeability methods; in vivo intestinal perfusion studies in humans; and in vivo or in situ intestinal perfusion studies in animals. In vitro permeation experiments can be conducted using excised human or animal intestinal tissue and in vitro permeation experiments can be conducted with epithelial cell monolayers. Alternatively, nonhuman systems capable of predicting the extent of drug absorption in humans can be used (e.g., in vitro epithelial cell culture methods). In the absence of evidence suggesting instability in the gastrointestinal tract, a drug is considered highly soluble when 90% or more of an administered dose, based on a mass determination or in comparison to an intravenous reference dose, is dissolved. A drug substance is considered highly permeable when the extent of absorption in humans is determined to be greater than 90% of an administered dose, based on mass-balance or in comparison to an intravenous reference dose. An IR drug product is considered rapidly dissolving when no less than 85% of the labeled amount of the drug substance dissolves within 30 minutes, using U.S. Pharmacopeia (USP) Apparatus I at 100 rpm (or Apparatus II at 50 rpm) in a volume of 900 ml or less in each of the following media: (1) 0.1 N HCl or Simulated Gastric Fluid USP without enzymes; (2) a pH 4.5 buffer; and (3) a pH 6.8 buffer or Simulated Intestinal Fluid USP without enzymes.

BCS Class I Drugs

Examples of BCS class I drugs include those listed in Kasim et al. Mol. Pharmaceutics 1(1): 85-96 (2004) and Lindenberger et al. Eur. J. Pharm. Biopharm. 58(2):265-78 (2004), such as amitriptyline hydrochloride, biperiden hydrochloride, chloroquine phosphate, chlorpheniramine maleate, chlorpromazine hydrochloride, clomiphene citrate, cloxacillin sodium, ergotamine tartrate, indinavir sulfate, levamisole hydrochloride, levothyroxine sodium, mefloquine hydrochloride, nelfinavir mesylate, neostigmine bromide, phenytoin sodium, prednisolone, promethazine hydrochloride, proguanil hydrochloride, quinine sulfate, salbutamol, warfarin sodium, caffeine, fluvastatin, Metoprolol tartrate, Propranolol, theophylline, verapamil, Diltiazem, Gabapentin, Levodopa, carbidopa, reserpine, ethynyl estradiol, norethindrone, saquinavir mesylate and Divalproex sodium.

Valacyclovir is an antiviral drug which is active against the Herpes viruses. It is used to treat infections with herpes zoster (shingles), herpes simplex genitalis (genital herpes), and herpes labialis (cold sores). Valacyclovir inhibits the replication of viral DNA which is necessary for viruses to reproduce themselves. Valacyclovir is converted to acyclovir in the body.

Gabapentin is a medication indicated as adjunctive therapy in the treatment of partial seizure in epilepsy and for the management of post-herpetic neuralgia (PHN). PHN is the pain that lasts one to three months after shingles has healed. Gabapentin is also used for the treatment of partial seizures in adults and children. Gabapentin is available in capsule, tablet, and oral solution forms. The mechanism of action of gabapentin is unknown, but it has been shown to display analgesic action and anticonvulsant activity. Despite being a Class I drug, gabapentin is not appreciably metabolized in humans. The bioavailability of gabapentin is not dosed proportionally; as the dose increases, the bioavailability of gabapentin decreases. At best, the bioavailability of gabapentin is 60% at a 900 mg dose, given three times a day. Food increases, only slightly, the rate and extent of absorption of gabapentin.

Levodopa is the “gold standard” for the treatment of Parkinson disease. The drug has a narrow absorption window and is absorbed mainly in the proximal small intestine. Gastric emptying of the drug plays an important role in its absorption. There are reports that clearly illustrate that “wearing off” and “on-off” phenomena are associated to the random fluctuation of levodopa levels in the Parkinson patients. Varying gastric emptying results in a considerable inter-subject variability and levels of levodopa need to be monitored to reduce the motor fluctuations.

Many BCS Class I drugs, such as verapamil, levadopa, metformin, and gabapentin, are absorbed only in the upper small intestine and have little or no absorption in the distal small intestine or colon. Many BCS Class I drugs require specific transport carriers in the intestinal tissue for delivery. These carriers can be saturated, thereby preventing absorption of the drug and resulting in sub-optimal absorption.

BCS Class II Drugs

Class II drugs are drugs that are particularly insoluble, or slow to dissolve, but that readily are absorbed from solution by the lining of the stomach and/or the intestine. Hence, prolonged exposure to the lining of the GI tract is required to achieve absorption. Such drugs are found in many therapeutic classes.

Many of the known Class II drugs are hydrophobic, and have historically been difficult to administer. Moreover, because of the hydrophobicity, there tends to be a significant variation in absorption depending on whether the patient is fed or fasted at the time of taking the drug. This in turn can affect the peak level of serum concentration, making calculation of dosage and dosing regimens more complex. Many of these drugs are also relatively inexpensive, so that simple formulation methods are required and some inefficiency in yield is acceptable.

In a preferred embodiment, the drug is intraconazole and its relatives fluoconazole, terconazole, ketoconazole, and saperconazole. Itraconazole is a Class II medicine used to treat fungal infections and is effective against a broad spetrum of fungi including dermatophytes (tinea infections), candida, malassezia, and chromoblastomycosis. Itraconazole works by destroying the cell wall and critical enzymes of yeast and other fungal infectious agents. Itraconazole can also decrease testosterone levels, which makes it useful in treating prostate cancer and can reduce the production of excessive adrenal corticosteroid hormones, which makes it useful for Cushing's syndrome. Itraconazole is available in capsule and oral solution form. For fungal infections the recommended dosage of oral capsules is 200-400 mg once a day.

Itraconazole has been available in capsule form since 1992, in oral solution form since 1997, and in an intravenous formulation since 1999. Since itraconazole is a highly lipophilic compound, it achieves high concentrations in fatty tissues and purulent exudates. However, its penetration into aqueous fluids is very limited. Gastric acidity and food heavily influence the absorption of the oral formulation (Bailey, et al., Pharmacotherapy, 10: 146-153 (1990)). The absorption of itraconazole oral capsule is variable and unpredictable, despite having a bioavailability of 55%.

Other Class II drugs include anti-infective drugs such as sulfasalazine, griseofulvin and related compounds such as griseoverdin; some anti malaria drugs (e.g. Atovaquone); immune system modulators (e.g. cyclosporine); and cardiovascular drugs (e.g. digoxin and spironolactone); and ibuprofen (analgesic); ritonavir, nevirapine, lopinavir (antiviral); clofazinine (leprostatic); diloxanide furoate (anti-amebic); glibenclamide (anti-diabetes); nifedipine (anti-anginal); spironolactone (diuretic); steroidal drugs such as Danazol; carbamazepine, and anti-virals such as acyclovir.

Danazol is derived from ethisterone and is a synthetic steroid. Danazol is designated as 17a-Pregna-2,4-dien-20-yno[2,3-d]-isoxazol-17-ol, has the formula of C₂₂H₂₇NO₂, and a molecular weight of 337.46. Danazol is used in the treatment of endometriosis, fibrocystic breast disease and hereditary angioedema. Danazol is administered orally, has a bioavailability that is not directly dose-related, and a half-life of 4-5 hours. Dosage increases in danazol are not proportional to increases in plasma concentrations. It has been shown that doubling the dose may yield only a 30-40% increase in plasma concentration. Danazol peak concentrations occur within 2 hours, but the therapeutic effect usually does not occur for approximately 6-8 weeks after taking daily doses.

Acyclovir is a synthetic nucleoside analogue that acts as an antiviral agent. Acyclovir is available for oral administration in capsule, tablet, and suspension forms. It is a white, crystalline powder designated as 2-amino-1,9-dihydro-9-[(2-hydroxyethoxy)methyl]-6H-purin-6-one, has an empirical formula of C₈H₁₁N₅O₃ and a molecular weight of 225. Acyclovir has an absolute bioavailability of 20% at a 200 mg dose given every 4 hours, with a half-life of 2.5 to 3.3 hours. The bioavailability decreases with increasing doses. Despite its low bioavailability, acyclovir is highly specific in its inhibitory activity of viruses due to its high affinity for thymidine kinase (TK) (encoded by the virus). TK converts acyclovir into a nucleotide analogue which prevents replication of viral DNA by inhibition and/or inactivation of the viral DNA polymerase, and through termination of the growing viral DNA chain.

Carbamazepine is used in the treatment of psychomotor epilepsy, and as an adjunct in the treatment of partial epilepsies. It can also relieve or diminish pain that is associated with trigeminal neuralgia. Carbamazepine given as a monotherapy or in combination with lithium or neuroleptics has also been found useful in the treatment of acute mania and the prophylactic treatment of bipolar disorders. Carbamazepine is a white to off-white powder, is designated as 5H-dibenz[b,f]azepine-5-carboxamide, and has a molecular weight of 236.77. It is practically insoluble in water and soluble in alcohol and acetone. The absorption of carbamazepine is relatively slow, despite a bioavailability of 89% for the tablet form. When taken in a single oral dose, the carbamazepine tablets and chewable tablets yield peak plasma concentrations of unchanged carbamazepine within 4 to 24 hours. The therapeutic range for the steady-state plasma concentration of carbamazepine generally lies between 4 and 10 mcg/mL.

BCS Class III and IV Drugs

Class III drugs have good water solubility and poor GI permeability and include proteins, peptides, polysaccharides, nucleic acids, nucleic acid oligomers and viruses. Examples of Class III drugs include abacavir sulfate, amiloride HCl, atropine sulfate, chloramphenicol, folic acid, hydrochlorthazide, lamivudine, methyldopa, mefloquine HCl, penicillamine, pyrazinamide, salbutamol sulfate, valproic acid, stavudine, ethosuximide, ergometrine maleate, colchicines, didanosine, cimetidine, ciprofloxacin, neomycin B, captopril, Atenolol, and Caspofungin.

Caspofungin is a Class III drug and is used to treat serious antifungal agents. Caspofungin acetate is a semisynthetic lipopeptide (echinocendin) compound synthesized from a fermentation product of Glarea lozoyensis. Caspofungin acetate is a hygroscopic, white to off-white powder, which is freely soluble in water and methanol, and slightly soluble in ethanol. The pH of a saturated aqueous solution of caspofungin acetate is approximately 6.6. Caspofungin acetate has an empirical formula of C₅₂H₈₈N₁₀O₁₅.2C2H₄O₂ and a formula weight of 1213.42. Caspofungin acetate is designated as 1-[(4R,5s)-5-[(2-aminoethyl)amino]-N²-(10,12-dimethyl-1-oxotetradecyl)-4-hydroxy-L-ornithine]-5-[(3IRI)-3-hydroxy-L-ornithine]pneumocandin B₀ diacetate (salt). Caspofungin acts through inhibition of the cell wall synthesis of fungi such as Aspergillus and Candida. Caspofungin acetate is currently available for intravenous injection at 50 mg/day with an elimination half-life of 9-10 hours and is suitable for once-daily regimens. Casposfungin is slowly metabolized by hydrolysis and N-acetylation and also undergoes spontaneous chemical degradation. The bioavailability of Caspofungin is currently 0%.

Class IV drugs are lipophilic drugs with poor GI permeability. Examples include acetazolamide, allopurinol, dapsone, doxycycline, paracetamol, nalidixic acid, clorothiazide, tobramycin, cyclosporin, tacrolimus, and paclitaxel.

Tacrolimus is a macrolide immunosuppressant produced by Streptomyces tsukubaensis. Tacrolimus prolongs the survival of the host and transplanted graft in animal transplant models of liver, kidney, heart, bone marrow, small bowel and pancreas, lung and trachea, skin, cornea, and limb. Tacrolimus acts as an immunosuppressant through inhibition of T-lymphocyte activation through a mechanism that is unknown. Tacrolimus has an empirical formula of C₄₄H₆₉NO₁₂.H₂O and a formula weight of 822.05. Tacrolimus appears as white crystals or crystalline powder. It is practically insoluble in water, freely soluble in ethanol, and very soluble in methanol and chloroform. Tacrolimus is available for oral administration as capsules or as a sterile solution for injection. Absorption of tacrolimus from the gastro-intestinal tract after oral administration is incomplete and variable. The absolute bioavailability of tacrolimus is approximately 17% at a 5 mg dose taken twice a day.

Paclitaxel is a chemotherapeutic agent that displays cytotoxic and antitumor activity. Paclitaxel is a natural product obtained via a semi-synthetic process from Taxus baccata. While having an unambiguous reputation of tremendous therapeutic potential, paclitaxel has some patient-related drawbacks as a therapeutic agent. These partly stem from its extremely low solubility in water, which makes it difficult to provide in suitable dosage form. Because of paclitaxel's poor aqueous solubility, the current approved (U.S. FDA) clinical formulation consists of a 6 mg/ml solution of paclitaxel in 50% polyoxyethylated castor oil (CREMOPHOR EL™) and 50% dehydrated alcohol. Am. J. Hosp. Pharm., 48:1520-24 (1991). In some instances, severe reactions, including hypersensitivity, occur in conjunction with the CREMOPHOR™ administered in conjunction with paclitaxel to compensate for its low water solubility. As a result of the incidence of hypersensitivity reactions to the commercial paclitaxel formulations and the potential for paclitaxel precipitation in the blood, the formulation must be infused over several hours. In addition, patients must be pretreated with steroids and antihistamines prior to the infusion. Paclitaxel is a white to off-white crystalline powder available in a nonaqueous solution for injection. It has an empirical formula of C₄₇H₅₁NO₁₄, and a molecular weight of 853.9. Paclitaxel is highly lipophilic and insoluble in water.

Both Class III and IV drugs are often problematic or unsuitable for sustained release or controlled release. Class III and Class IV drugs are characterized by biomembrane permeability and are commonly delivered parenterally. Traditional approaches to parenteral delivery of poorly soluble drugs include using large volumes of aqueous diluents, solubilizing agents, detergents, non-aqueous solvents, or non-physiological pH solutions. These formulations, however, can increase the systemic toxicity of the drug composition or damage body tissues at the site of administration.

Ulcer Formulations

Helicobacter pylori infection is a major pathogenic factor in gastro-duodenal diseases, including chronic active gastritis, peptic ulcers and gastric neoplasia (Goldblum, et al., Am. J. Gastroenterol, 97: 302-311 (2002)). Treatment regimens aimed at bacterial cure are recommended for patients with symptomatic H. pylori infection (NIH Consensus Conference. J. Am. Med. Assoc., 272: 65-69 (1994)). The Maastricht 2000 Consensus Report on the management of H. pylori recommends the use of proton pump inhibitors (PPI) or ranitidine bismuth citrate (RBC) with clarithromycin and amoxicillin as first-line treatment in primary care (Malfertheiner, et al., Aliment. Pharmacol. Ther. 16:167-180 (2002)).

H. pylori lives in close proximity to the gastric epithelial surface, deeply buried within the mucus gel (Hussy, et al., Gut, 31:134-138 (1990)). Mucus gel is a complex mixture of water, glycoprotein, proteins and lipids that form a highly viscous layer that covers the surface of the gastric mucosa and severely restricts the diffusion of hydrogen and macromolecules (Allen, et al., “Structure and function of gastrointestinal mucus”, In: Physiology of the Gastrointestinal Tract. New York: Raven. 617-639 (1981)). The mechanisms by which gastric mucus reduces the diffusion of antibiotics are not clearly understood. It has been suggested that gastric mucus forms a dense gel matrix holding an unstirred water layer within its interstices thus limiting antibiotic transfer (Allen, et al., Physiology Rev., 73: 823-857 (1993)). In order for any antibiotic to work effectively, antibiotics have to penetrate the mucus layer to eradicate the H. pylori. Since 95% of the gastric mucus is water, held in the dispersed strands of branching mucin glycoprotein molecules, solubilization of any antibiotic must occur to allow its diffusion into the water-filled pores established by the extended carbohydrate chains. Moreover mucus viscosity increases when the pH is lowered from 7 to 2 (Backer, et al., Am. J. Physiol., 261: G827-832 (1991)), which may explain why antibiotic eradication therapy only works well with the concomitant acid suppression therapy, rendering gastric mucus less viscous and easy to permeate. Furthermore, acid suppression prevents degradation of a number of H. pylori sensitive antibiotics like clarithromycin, which is acid labile and shows more than a 16-fold decrease in activity at pH 5.5 (Cederbrant, et al., J. Antimicrob. Chemother., 34: 1025-1029 (1994)). This is the reason why a single agent, which does not have an adequate concentration to bacteria ratio within the mucous layer, does not cause sufficient eradication, and often results in increased resistance to H. pylori (Axon and Scand, J. Gastroenterol., 29: 16-23 (1994)).

A majority of the antimicrobial drugs belong to “Class II” of the Biopharmaceutics Classification System (BCS). Representative antibiotics to kill Helicobacter pylori include amoxicillin, tetracyline and metronidazole. Clarithromycin can be substituted for the 15-25% of people whose infections are resistant to metronidazole. In the preferred embodiment, other therapeutic agents including acid suppressants (H₂ blockers include cimetidine, ranitidine, famotidine, and nizatidine; Proton pump inhibitors include omeprazole, lansoprazole, rabeprazole, esomeprazole, and pantoprozole), mucosal defense enhancing agent (bismuth salts; bismuth subsalicylate) and/or mucolytic agents (megaldrate).

B. Formulations

Controlled Release Materials

For drugs requiring absorption in buccal and sublingual regions of the GIT, bioadhesive tablets and particularly bioadhesive multiparticulates and nanoparticles are desirable. Drugs absorbed in these sites avoid first-pass metabolism by liver and degradation by GIT enzymes and harsh pH conditions typically present in the stomach and small intestine. Drugs absorbed in the buccal and sublingual compartments benefit from rapid onset of absorption, typically within minutes of dosing. Particularly suitable are bioadhesive particulates in fast-dissolving dosage forms, eg, OraSolv (Cima Labs) that disintegrate within 30 sec after dosing and release the bioadhesive particules. Target release profiles include immediate release (IR) and combinations of zero-order controlled release (CR) kinetics and first-order CR kinetics.

For drugs requiring absorption in the stomach and upper small intestine and/or topical delivery to these sites, particularly drugs with narrow absorption windows, bioadhesive, gastroretentive drug delivery systems are the option of choice. Bioadhesive tablets and multiparticulates are formulated to reside for durations greater than 3 hrs and optimally greater than 6 hrs in the fed state. Drug release profiles from these systems are tailored to match the gastric residence times, so that greater than 85% of the encapsulated drug is released during the gastric residence time. Target release profiles include zero-order CR kinetics, first-order CR kinetics and combinations of IR and CR kinetics.

For drugs requiring absorption or topical delivery only in the small intestine, enteric-coated, bioadhesive drug delivery systems are preferred method. Such systems are particularly well suited for topical delivery of therapeutics to Crohn's disease patients. Enteric-coated, bioadhesive tablets and multiparticulates are formulated to reside in the stomach for durations less than 3 hrs in the fed state and less than 1 hr in the fasted state, during which time less than 10% of the encapsulated drug is released, due to the enteric coating.

Following gastric emptying, the enteric coating is “triggered” to dissipate, revealing the underlying bioadhesive coating. Suitable triggers include pH and time duration. Typical of enteric polymers utilizing pH as a trigger are Eudragit polymers manufactured by Rohm America: Eudragit L100-55 dissolves at pH values greater than 5.5, typically found in duodenum; Eudragit L100 dissolves at pH values exceeding 6.0, typically found in jejunum; Eudragit S100 dissolves at pH values exceeding 7.0, typically found in ileum and the ileocecal junction. Also suitable are cellulosic enteric polymers such as cellulose acetate phthalate.

Time may be used as a trigger to unmask the bioadhesive coating. Coatings that dissolve after 3 hrs when the dosage form is administered in the fed state and after 1-2 hrs when the dosage form is administered in the fasted state are suitable for bioadhesive delivery systems to small intestine. Erosion of soluble polymer layers is one means to achieve a time-triggered, enteric dissolution. Polymers such as HPMC, HPC, PVP, PVA or combinations of the above may be used as time-delayed, enteric coatings and applying thicker coating weights can increase timing of the dissolution of the coating. Suitable enteric coating materials are shown in Table 2.

TABLE 2
Methacrylate-based coating materials

| Functionality | Trade name |
|---|---|
| Anionic polymer of | Eudragit ® L 100-55 - powder, spray dried L 30 D-55 |
| methacrylic acid and | which can be reconstituted for targeted delivery in the |
| methacrylates with a - COOH | duodenum |
| group | Eudragit ® L 30 D-55 - aqueous dispersion, pH dependent |
| | polymer soluble above pH 5.5 for targeted delivery in the |
| | duodenum |
| | Eudragit ® L 100 - powder, pH dependent polymer soluble |
| | above pH 6.0 for targeted delivery in the jejunum |
| | Eudragit ® S 100 - powder, pH dependent polymer |
| | soluble above pH 7.0 for targeted delivery in the ileum. |
| | Eudragit ® FS 30 D - aqueous dispersion, pH dependent |
| | polymer soluble above pH 7.0, requires no plasticizer |
| Cationic polymer with a | Eudragit E 100 - granules, pH dependent, soluble in |
| dimethylaminoethyl | gastric fluid up to 5.0, swellable and permeable above pH 5.0. |
| ammonium group | Eudragit ® E PO - powder form of E-100 |
| Copolymers of acrylate | Insoluble, High Permeability |
| and methacrylates with | Eudragit ® RL 30D - aqueous dispersion, pH independent |
| quarternary ammonium | polymer for sustained release formulations |
| group. | Eudragit ® RL PO - powder, pH independent polymer for |
| | matrix formulations |
| | Eudragit ® RL 100 - granules, pH independent |
| | Insoluble, Low Permeability |
| | Eudragit ® RS 30D - aqueous dispersion, pH independent |
| | polymer for sustained release formulations |
| | Eudragit ® RS PO - powder, pH independent polymer |
| | for matrix formulations |
| | Eudragit ® RS 100 - granules, pH independent |
| Copolymers of acrylate | Eudragit RD 100 - powder, pH independent for fast |
| and methacrylates with | disintegrating films |
| quarternary ammonium | |
| group in combination with | |
| sodium | |
| carboxymethylcellulose | |

Alternately, non-permeable coatings of insoluble polymers, eg, cellulose acetate, ethylcellulose, can be used as enteric coatings for delayed/modified release (DR/MR) by inclusion of soluble pore formers in the coating, eg PEG, PVA, sugars, salts, detergents, Triethyl Citrate, Triacetin etc at levels ranging from 0.5 to 50% w/w of the coating and most preferably from 5 to 25% w/w of the coating.

Also suitable are rupturable coating systems, eg, Pulsincap®, that use osmotic forces of swelling from hydrophilic polymers to rupture enteric membranes to reveal underlying bioadhesive coatings.

Target release profiles include: no more than 10% drug release during the first 3 hrs post-dosing followed by either IR kinetics, zero-order CR kinetics, first-order CR kinetics and combinations of IR and CR kinetics.

In the preferred embodiment of an ulcer treating formulation, the formulation includes a multilayer core enveloped by a bioadhesive coating. In the aqueous environment of the gastro-intestinal tract (GIT), the bioadhesive coating slightly swells and adheres to the mucosa. As a result, the drug formulation enhances the bioavailability of the therapeutics agents through increased residence time at the target absorption site. The capsule shape tablet is partially enveloped in a bioadhesive, mucoadhesive polymeric plug such that the drug layer-ends remain exposed for drug release. The first layer may contain amoxicillin, clarithromycin or other antimicrobial agents such as metronidazole that allows these agents to be released in a variety of manners. The second layer contains an anti-secretory agent such as proton pump inhibitors (PPI) or other drugs of different classes such as ranitidine bismuth citrate (RBS). This PPI agent or antimicrobial agent may be enteric coated to prevent its degradation in the stomach. The first layer may also contain antacids to raise the pH of the stomach content so that there is no degradation of these agents in the stomach. The formulation may also include mucolytic agents, mucosal protection agent promoters, anti-gastrin agents, bismuth preparations, and H-2 receptor antagonists. The coatings may be applied using a variety of techniques including fluidized-bed coating, pan-coating, dip-coating, and extrusion.

For drugs requiring absorption or topical delivery only in the lower small intestine and colon enteric-coated, bioadhesive drug delivery systems are preferred method. Such systems are particularly well suited for topical delivery of therapeutics to patients with Inflammatory Bowel Disease (IBD) including Crohn's disease and Ulcerative Colitis. Enteric-coated, bioadhesive tablets and multiparticulates are formulated to reside in the stomach for durations less than 3 hrs in the fed state and less than 1 hr in the fasted state, during which time less than 10% of the encapsulated drug is released, due to the enteric coating.

Following gastric emptying, the enteric coating is “triggered” to dissipate, revealing the underlying bioadhesive coating. Suitable triggers include pH, time duration and enzymatic action of colonic bacteria. Typical of enteric polymers for delivery to lower GIT utilizing pH as a trigger are Eudragit polymers manufactured by Rohm America: Eudragit S100 and FS dissolves at pH values exceeding 7.0, typically found in ileum and the ileocecal junction.

Time may be used as a trigger to unmask the bioadhesive coating. Coatings that dissolve after 4-5 hrs when the dosage form is administered in the fasted state and after 5-8 hrs when the dosage form is administered in the fed state are suitable for bioadhesive delivery systems to lower small intestine and colon. Erosion of soluble polymer layers is one means to achieve a time-triggered, enteric dissolution. Polymers such as HPMC, HPC, PVP, PVA or combinations of the above may be used as time-delayed, enteric coatings and timing of the dissolution of the coating can be increased by applying thicker coating weights.

Alternately, non-permeable coatings of insoluble polymers, eg, cellulose acetate, ethylcellulose, can be used as enteric coatings for delayed/modified release (DR/MR) by inclusion of soluble pore formers in the coating, eg PEG, PVA, sugars, salts, detergents, Triethyl Citrate, Triacetin etc at levels ranging from 0.5 to 50% w/w of the coating and most preferably from 5 to 25% w/w of the coating.

Also, coatings of polymers that are susceptible to enzymatic cleavage by colonic bacteria are another means of ensuring release to distal ileum and ascending colon. Materials such as calcium pectinate can be applied as coatings to tablets and multiparticulates and disintegrate in the lower GIT, due to bacterial action. Calcium pectinate capsules for encapsulation of bioadhesive multiparticulates are also available.

Target release profiles include: no more than 10% drug release during the first 4-5 hrs (fasted state) and 5-8 hrs (fed state) hrs post-dosing followed by either IR kinetics, zero-order CR kinetics, first-order CR kinetics and combinations of IR and CR kinetics.

Bioadhesive Materials

Bioadhesives are included in the formulation to improve gastrointestinal retention via adherence of the formulation to the walls of the GI tract. As used herein “bioadhesive” generally refers to the ability of a material to adhere to a biological surface for an extended period of time. Bioadhesion requires contact between a bioadhesive material and a surface (e.g. tissue and/or cells). Thus the amount of bioadhesive force is affected by both the nature of the bioadhesive material, such as a polymer, and the nature of the surrounding medium. The bioadhesive materials described herein may be used in a wide variety of drug delivery and diagnostic applications. Bioadhesive materials may be formed into microparticles, such as microspheres or microcapsules, or may be a coating on such microparticles. In the preferred embodiment, the material is applied as a coating to any longitudinally compressed tablet.

Bioadhesive polymers are described in U.S. Pat. No. 6,235,313 to Mathiowitz et al. Suitable polymers include polylactic acid (2 kDa MW, types SE and HM), polystyrene, poly(bis carboxy phenoxy propane-co-sebacic anhydride) (20:80) (poly (CCP:SA)), alginate (freshly prepared); and poly(fumaric anhydride-co-sebacic anhydride (20:80) (p[FA:SA]), types A (containing sudan red dye) and B (undyed). Other high-adhesion polymers include p[FA:SA] (50:50) and non-water-soluble polyacrylates and polyacrylamides. In designing bioadhesive polymeric formulations based on polylactides, polymers that have high concentrations of carboxylic acid are preferred. This can be accomplished by using low molecular weight polymers (Mw 2000), since low molecular weight polymers contain high concentration of carboxylic acids at the end groups.

In a preferred embodiment, bioadhesive polymers are typically hydrophobic enough to be non-water-soluble, but contain a sufficient amount of exposed surface carboxyl groups to promote adhesiveness. These include, among others, non-water-soluble polyacrylates and polymethacrylates; polymers of hydroxy acids, such as polylactide and polyglycolide; polyanhydrides; polyorthoesters; blends comprising these polymers; and copolymers comprising the monomers of these polymers. Blending or copolymerization sufficient to provide a certain amount of hydrophilic character can be useful to improve wettability of the materials. For example, about 5% to about 20% of monomers may be hydrophilic monomers. Preferably, the polymers are bioerodable, with preferred molecular weights ranging from 1000 to 50,000 Da, and most preferably 2000 to 20,000 Da.

Polyanhydrides are a preferred type of bioadhesive polymer. The use of certain bioadhesive polymers, particularly polyanhydrides, allows one polymer additive to serve several functions simultaneously to enhance oral uptake. Suitable polyanhydrides include polyadipic anhydride (“p(AA)”), polyfumaric anhydride, polysebacic anhydride, polymaleic anhydride, polymalic anhydride, polyphthalic anhydride, polyisophthalic anhydride, polyaspartic anhydride, polyterephthalic anhydride, polyisophthalic anhydride, poly carboxyphenoxypropane anhydride and copolymers with other polyanhydrides at different mole ratios. p(AA) is a surface-eroding polymer belonging to the polyanhydride family of bioerodable and biocompatible polymers. The polymer is a low molecular weight (2-8 kDa) thermoplastic polymer that quickly degrades to adipic acid monomer and adipic anhydride (both of which are considered GRAS for food applications) over the course of 24 hrs at physiological pH.

Optionally, the polymer is a blend of hydrophilic polymers and bioadhesive hydrophobic polymers. Suitable hydrophilic polymers include hydroxypropylmethylcellulose, hydroxypropylcellulose, carboxymethylcellulose, polyvinylalcohols, polyvinylpyrollidones, and polyethylene glycols. The hydrophobic polymer may contain gastrosoluble polymers that dissolve in stomach contents, such as Eudragit® E100. The hydrophobic polymer may contain entero-soluble materials that dissolve in the intestine above pH 4.5, such as Eudragit® L-100, Eudragit® S-100, cellulose acetate phthalate, hydroxypropyl methylcellulose phthalate, hydroxypropyl methylcellulose acetate succinate, Eastacryl® 30D dispersion from Eastman Chemicals, Sureteric® (polyvinyl acetate phthalate) and Acryl Eze®.

In a preferred embodiment, the bioadhesive polymers contain a water insoluble hydrophobic backbone and nucleophilic functional groups. A compound containing an aromatic group which contains one or more hydroxyl groups, such as catechol, can be grafted onto a polymer or coupled to individual monomers. The polymer or monomer that forms the polymeric backbone may contain accessible functional groups that easily react with molecules contained in the aromatic compounds, such as amines and thiols. In a preferred embodiment, the polymer contains amino reactive moieties, such as aldehydes, ketones, carboxylic acid derivatives, cyclic anhydrides, alkyl halides, acyl azides, isocyanates, isothiocyanates, and succinimidyl esters.

Polymers that contain a catechol functionality are bioadhesive. “Catechol” refers to a compound with a molecular formula of C₆H₆O₂ and the following structure: 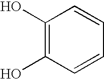
These aromatic groups are substituted for monomers on the backbone of a suitable polymer. The degree of substitution varies based on the desired adhesive strength. It may be as low as 10%, 25%, 50%, or up to 100% substitution. On average, at least 20% of the monomers in a suitable polymeric backbone are substituted with at least one aromatic group. These polymers are available from Spherics, Inc., RI as Spheromers™.

In a preferred embodiment, the aromatic compound containing one or more hydroxyl groups is catechol or a derivative thereof. Optionally the aromatic compound is a polyhydroxy aromatic compound, such as a trihydroxy aromatic compound (e.g. phloroglucinol, benserazide) or a multihydroxy aromatic compound (e.g. tannin). The catechol derivative may contain a reactive group, such as an amino, thiol, or halide group. The preferred catechol derivative is 3,4-dihydroxyphenylalanine (DOPA), which contains a primary amine. Tyrosine, the immediate precursor of DOPA, which differs only by the absence of one hydroxyl group in the aromatic ring, can also be used. Tyrosine is capable of conversion (e.g. by hydroxylation) to the DOPA form.

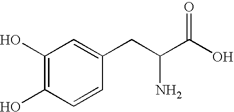

### 3,4-dihydroxyphenylalanine

In another preferred embodiment, the aromatic compound is an amine-containing aromatic compound, such as an amine-containing catechol derivative.

DOPA-containing mucoadhesive polymers include DOPA-maleic anhydride co-polymer, isopthalic anhydride polymer, DOPA-methacrylate polymers, DOPA-cellulosic based polymers, and DOPA-acrylic acid polymers.

Excipents may also be added to improve mucoadhesion. Suitable excipients include FeO/Fe₂O₃, fumaric anhydride oligomer (FAO), L-DOPA-L-DOPA dimer, and adipic anhydride pre-polymer (AAP).

Bioadhesive materials available from Spherics, Inc., Lincoln, R.I., include Spheromer™ I (poly(fumaric acid:sebacic acid) (p[DA:SA])”, as described in U.S. Pat. No. 5,955,096 to Mathiowitz et al.), Spheromer™ II (anhydride oligomers, such as Fumaric Anhydride Oligomer and Metal oxides, such as CaO, ferric oxide, magnesium oxide, titanium dioxide, as described in U.S. Pat. No. 5,985,312 to Jacob et al.), and Spheromer™ III (L-DOPA grafted onto butadiene maleic anhydride at approximately 20% substitution efficiency (L-DOPA-BMA)). Spheromer™ II may be blended with methylmethacrylates, celluloses and substituted celluloses, polyvinylpyrollidones, PEGs, Poly(vinyl alcohols). Alternatively Spheromer™ II may be blended with other bioadhesive polymers including p[FA:SA], p(AA), and L-DOPA-BMA.

Excipients

The active compounds (or pharmaceutically acceptable salts thereof) may be administered in a formulation wherein the active compound(s) is in an admixture with one or more pharmaceutically acceptable carriers, excipients or diluents. The pharmaceutical formulations may be produced using standard procedures.

The compounds may be complexed with other agents as part of the formulation. The pharmaceutical compositions may take the form of, for example, tablets or capsules prepared by conventional means with pharmaceutically acceptable excipients such as binding agents (e.g., acacia, methylcellulose, sodium carboxymethylcellulose, polyvinylpyrrolidone (Povidone), hydroxypropyl methylcellulose (HPMC), sucrose, starch, and ethylcellulose); fillers (e.g., corn starch, gelatin, lactose, acacia, sucrose, microcrystalline cellulose, kaolin, mannitol, dicalcium phosphate, calcium carbonate, sodium chloride, or alginic acid); lubricants (e.g. magnesium stearates, stearic acid, silicone fluid, talc, waxes, oils, and colloidal silica); and disintegrators (e.g. micro-crystalline cellulose, corn starch, sodium starch glycolate and alginic acid. If water-soluble, such formulated complexes may then be dissolved in an appropriate buffer, for example, phosphate buffered saline or other physiologically compatible solutions. Alternatively, if the resulting complex has poor solubility in aqueous solvents, then it may be formulated with a surfactant such as TWEEN™, or polyethylene glycol, sodium lauryl sulfate, sodium caprate, pluronics, Span 80 and lecithin. Thus, the compounds and their physiologically acceptable solvates may be formulated for administration.

Excipents may also be added to the bioadhesive polymeric composition to alter its porosity and permeability. Suitable excipients may include inorganic and organic materials such as sucrose, hydroxypropyl cellulose, sodium chloride, sodium chloride, xylitol, sorbitol, lactose, dextrose, maltodextrins and dextrates

Excipents may also be added to the bioadhesive polymeric composition to alter its hydration and disintegration properties. Suitable pH dependent enteric excipients may include cellulose acetate phthalate.

Excipents may also be added as a “wicking agent” to regulate the hydration of the bioadhesive polymeric composition. Suitable excipients may include acdisol, hydroxypropyl cellulose, hydroxypropyl methyl cellulose, cellulose acetate phthalate.

p(AA) prevents coalescence of drug domains within the spray-dried product resulting in increased drug surface area available for dissolution. Additionally, adipic acid monomer generated during polymer degradation increases acidity in the microenvironment of the spray-dried drug particle. By changing the pH, some of the drugs may become more soluble.

Blending or copolymerization sufficient to provide a certain amount of hydrophilic character can be useful to improve wettability of the materials. For example, about 5% to about 20% of monomers may be hydrophilic monomers. Hydrophilic polymers such as hydroxylpropylcellulose (HPC), hydroxpropylmethylcellulose (HPMC), carboxymethylcellulose (CMC) are commonly used for this purpose.

The drugs may optionally be encapsulated or molecularly dispersed in polymers to reduce particle size and increase dissolution. The polymers may include polyesters such as poly (lactic acid) or P[LA], polycaprylactone, polylactide-coglycolide or P[LGA], poly hydroxybutyrate poly β-malic acid); polyanhydrides such as poly(adipic)anhydride or P(AA), poly (fumaric-co-sebacic)anhydride or p[FA:SA], poly(sebacic)anhydride or P(SA); cellulosic polymers such as ethylcellulose, cellulose acetate, cellulose acetate phthalate, etc; acrylate and methacrylate polymers such as Eudragit RS 100, RL 100, E100 PO, L100-55, L100, S100 (distributed by Rohm America) or other polymers commonly used for encapsulation for pharmaceutical purposes and known to those skilled in the art. Also suitable are hydrophobic polymers such as polyimides.

The system can also be designed to extend the time period for release by increasing the drug to polymer ratio, with release drawn out to 80% in 90 minutes (in vitro). Increased relative drug concentration is believed to have the effect of increasing the effective drug domain size within the polymer matrix; and increased drug domain size results in slower drug dissolution. In the case of a polymer matrix containing certain types of hydrophobic polymers, the polymer will act as a bioadhesive material and increase the retention time of the drug product in the gastrointestinal tract. Delayed release and extended release compositions can be obtained by complexing drug with a pharmaceutically acceptable ion-exchange resin and coating such complexes. The formulations are coated with a substance that will act as a barrier to control the diffusion of the drug from its core complex into the gastrointestinal fluids. Optionally, the formulation is coated with a film of a polymer which is insoluble in the acid environment of the stomach, and soluble in the basic environment of lower GI tract in order to obtain a final dosage form that releases less than 10% of the drug dose within the stomach.

As discussed above, examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, methacrylic resins, zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the tablets, multiparticulate formulations, beads, granules, or particles include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet, multiparticulate, bead, or granule remains intact during storage and until administration. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-1 50 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, Pluronics, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine lauryl sulfobetaine, and lecithin.

If desired, the tablets, beads, granules, or particles may also contain minor amounts of nontoxic auxiliary substances, such as wetting or emulsifying agents, dyes, pH buffering agents, or preservatives.

The BCS Class II and IV drugs may optionally be encapsulated or molecularly dispersed in polymers to reduce particle size. The polymers may include polyesters such as poly(lactic acid) or P[LA], polycaprylactone, poly(lactide-co-glycolide) or P[LGA], polyhydroxybutyrate poly(β-malic acid); polyanhydrides such as poly(adipic)anhydride or P(AA), poly(fumaric-co-sebacic)anhydride or p[FA:SA], poly(sebacic)anhydride or P(SA); cellulosic polymers such as ethylcellulose, cellulose acetate, cellulose acetate phthalate, etc; acrylate and methacrylate polymers such as Eudragit RS 100, RL 100, E100 PO, L100-55, L100, S100 (distributed by Rohm America) or other polymers commonly used for encapsulation for pharmaceutical purposes and known to those skilled in the art.

II. Methods of Making the Formulations

Solid oral dosage forms are typically prepared by blending powder drug or drug particles (i.e. drug in micro or nanoparticles) with excipients such as those discussed above and compressing the mixture into the form of a tablet. Alternately the mixture may be incorporated into standard pharmaceutical dosage forms such as gelatin capsules and tablets. Gelatin capsules, available in sizes 000, 00, 0, 1, 2, 3, 4, and 5, from manufactures such as Capsugel®, may be filled with mixtures and administered orally. Similarly, macrospheres may be dry blended or wet-granulated with diluents such as microcrystalline cellulose, lactose, cabosil and binders such as hydroxypropylmethylcellulose, hydroxypropylcellulose, carboxymethylcellulose and directly compressed to form tablets. The dimensions of the tablets are limited only by the engineering of dies available for tabletting machines. Dies to form tablets in round, oblong, convex, flat, and bullet designs in sizes ranging from 1 to 20 mm are available. The resulting tablets may weigh from 1 to 5,000 mg and carry macrospheres at loadings of 1 to 80% w/w.

The resulting tablets may be coated with sugars, enteric polymers or gelatin to alter dissolution of the tablet. Premature dissolution of the tablet in the mouth may be prevented by coating with hydrophilic polymers, such as hydroxypropylmethylcellulose or gelatin, resulting in dissolution in the stomach.

The tablet or solid oral dosage form may optionally contain absorption enhancers including: sodium caprate, ethylenediamine tetra(acetic acid) (EDTA), Lutrols, polysorbates, sodium lauryl sulfate, citric acid, lauroylcarnitine, palmitoylcarnitine, tartaric acid, Vitamin E TPGS and other agents known to increase GI permeability by affecting integrity of tight junctions.

Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980). The formulation may be in the form of a tablet, capsule, minitab, filled tablet, multiunit filled capsules, multiunits embedded in a rapidly disintegrating tablet, osmotic device, slurry, dispersion, or suspension. In the preferred embodiment, the formulation is a solid oral dosage formulation, such as a tablet, multiparticulate composition, or capsule.

The drug may be incorporated into a polymer matrix by spray drying at any appropriate loading, such as from 1 to 90% w/w, from 1 to 50% w/w, 20 to 70% w/w, from 40 to 60% w/w, and preferably in a range from 20% to 30% w/w. Using other processes such as hot melt extrusion, high shear mixing, the drug loading may vary from 20% to 90% and most preferably from 50-70%.

Drug release rates may be controlled by varying the proportion of drug to carrier in the solution used to prepare the formulation. For example, in some formulations, a drug-polyanhydride system can release drug rapidly, with at least 40% of the drug load in 30 minutes and at least 70% in 60 minutes (in vitro). Drugs are incorporated into the polymer matrix at loadings of 1 to 50% w/w and most preferably in the range of 20-30% w/w.

The system can also be designed to extend the time period for release by increasing the drug to carrier ratio, with release drawn out to 80% in 90 minutes (in vitro). Increased relative drug concentration is believed to have the effect of increasing the effective drug domain size within a polymer matrix; and increased drug domain size results in slower drug dissolution. In the case of a polymer matrix containing certain types of hydrophobic polymers, the polymer will act as a bioadhesive material and increase the retention time of the dosage form in the gastrointestinal tract. Increased drug dissolution rates combined with the bioadhesive properties of the polymer matrix results in (1) increased uptake of the drug and (2) reduction in differences found in the fed and fasted states for BCS Class I drugs.

A. Formation of Drug-Polymer Particles

The drug-polymer matrices may be fabricated using any of the encapsulation methods known to those skilled in the art, including but not limited to: solvent evaporation, solvent removal, spray-drying, phase-inversion encapsulation, spontaneous emulsification, coacervation, hot melt encapsulation, hot melt extrusion, spray-congealing, prilling and grinding. It is understood that the drug-polymer products may be further processed into oral dosage form using any of the standard pharmaceutical techniques including but not limited to tabletting, extrusion-spheronization, hot melt extrusion and fluidized bed coating for multiparticulate dosage forms and capsule-filling.

Because the primary source of adhesiveness and of prevention of aggregation is the nature of the polymer(s) forming the microspheres, the exact method of preparation is critical. The preferred method is spray drying of a solution in which the polymer and the drug are dissolved due to its simplicity. Other suitable methods include spray drying of a solution containing dissolved polymer and dispersed fine particles of drug or freeze-drying of a solution containing dissolved polymer and dissolved or suspended drug. Another method involves dissolving a polymer and dissolving or suspending a drug, and then diluting with a large volume (5× to 20×, for example) of a non-solvent for the polymer and the drug, where the solvent is substantially miscible with the non-solvent (at 20×, at least about 8 to 10% soluble). In preferred pairs of solvents and non-solvents, the absolute values of the differences in solubility parameter “delta” between the solvent and the non-solvent is less than about six. (Delta has units of square root of [calories/cm³]).

The resulting particles are suitable for capsules, tableting and other conventional dosage forms.

Spray Drying

In one embodiment, the composition contains a drug/polymer mixture co-dissolved in a mutual solvent and then spray-dried to form microparticles in the range of 2-100 μm in diameter. Drug loadings can range from 0.5-60% (w/w) drug with polymer, but are typically in the range of about 30% to 40%. Polymer systems contain polymers with bioadhesive qualities, and in the preferred embodiment may include either pure polyanhydride polymers, or mixtures of other biocompatible polymers (e.g., methacrylates, polyesters, polysaccharides) with polyanhydrides. The polymer system acts as a matrix for more rapid dissolution of the drug due to increased surface area by maintaining the micronized drug particle size. Spray dried polymer/drug product is then incorporated with suitable pharmaceutical excipients in a capsule oral dose form or may be filled into a softgelatin capsule after suspending in suitable vehicle.

Solvent Evaporation

In this method the polymer is dissolved in a volatile organic solvent, such as methylene chloride. The drug (either soluble or dispersed as fine particles) is added to the solution, and the mixture is suspended in an aqueous solution that contains a surface active agent such as poly(vinyl alcohol). The resulting emulsion is stirred until most of the organic solvent is evaporated, leaving solid particles. Several different polymer concentrations can be used, including concentrations ranging from 0.05 to 0.20 g/ml. The solution is loaded with a drug and suspended in 200 ml of vigorously stirred distilled water containing 1% (w/v) poly(vinyl alcohol) (Sigma). After 4 hours of stirring, the organic solvent evaporates from the polymer, and the resulting particles are washed with water and dried overnight in a lyophilizer. Particles with different sizes (1-1000 microns) and morphologies can be obtained by this method. This method is useful for relatively stable polymers like polyesters and polystyrene.

However, labile polymers, such as polyanhydrides, may degrade during the fabrication process due to the presence of water. For these polymers, the following two methods, which are performed in completely anhydrous organic solvents, are more useful.

Hot Melt Microencapsulation

This is a thermal processing method in which drug, homogeneously distributed in a polymeric matrix, is forced through a die under controlled conditions. Intense mixing and agitation during processing results in a more uniform dispersion of fine drug particles (Drug Dev. Ind. Pharmacy, Vol 28, issue 7, pp 757, 2003). This method offer the advantages for making spherical pellets, granules, films as well as tablets. The processing includes either a single or twin rotating screw extruder. Depending upon the physical and chemical properties of the drug and other excipients, the drug may be present as undissolved particles, a solid solution or a combination. Plasticizers, anti-oxidants, release controlling agents can be included to improve the processing conditions and stability of the matrix forming bioadhesive polymer. The plasticizers may be solid or liquid in nature.

In one embodiment, the polymer is first melted and then mixed with the solid particles of dye or drug that have been sieved to less than 50 microns. The mixture is suspended in a non-miscible solvent like silicon oil, and, with continuous stirring, heated to 5° C. above the melting point of the polymer. Once the emulsion is stabilized, it is cooled until the polymer particles solidify. The resulting particles are washed by decantation with petroleum ether to give a free-flowing powder. Particles with sizes between one to 1000 microns are obtained with this method. The external surfaces of spheres prepared with this technique are usually smooth and dense. This procedure is used to prepare particles made of polyesters and polyanhydrides. However, this method is limited to polymers with molecular weights between 1000 and 50,000 Da.

Solvent Removal

This technique is primarily designed for polyanhydrides. In this method, the drug is dispersed or dissolved in a solution of the selected polymer in a volatile organic solvent like methylene chloride. This mixture is suspended by stirring in an organic oil (such as silicon oil) to form an emulsion. Unlike solvent evaporation, this method can be used to make particles from polymers with high melting points and different molecular weights. Particles that range between 1-300 microns can be obtained by this procedure. The external morphology of spheres produced with this technique is highly dependent on the type of polymer used.

Extrusion-Spheronization

Core particles may be prepared by the process of granulation-extrusion-spheronization. In this process, micronized drug is mixed with microcrystalline cellulose, binders, diluents and water and extruded as a wet mass through a screen. The result is rods with diameters equal to the opening of the extrusion screen, typically in the size range of 0.1 to 5 mm. The rods are then cut into segments of approximately equal length with a rotating blade and transferred to a spheronizer. The spheronizer consists of a rapidly rotating, textured plate which propels rod segments against the stationary walls of the apparatus. Over the course of 1-10 minutes of spheronization, the rods are slowly transformed into spherical shapes by abrasion. The resulting spheroid cores are then discharged from the machine and dried at 40-50° C. for 24-48 hours using tray-driers or fluidized bed dryers. The cores may then be coated with rate-releasing, enteric or bioadhesive polymers using either pan-coating or fluidized-bed coating devices.

B. Preferred Delivery Systems

Tablets, capsules and multi-layer devices can be formulated to produce the desired release and uptake. One can create different release rates for one drug or a combination of drugs by changing the composition of the particulate cores, the relative population of particulate cores containing different drugs or having different formulations, or the type and level of rate controlling polymers as well as the bioadhesive polymer composition coating the particulates.

Tri-layer tablets provide first-order and, more advantageously, zero-order, release profiles. It is possible to create different release rates for drug by changing the composition of the core matrix, as well as the coating and outer layers.

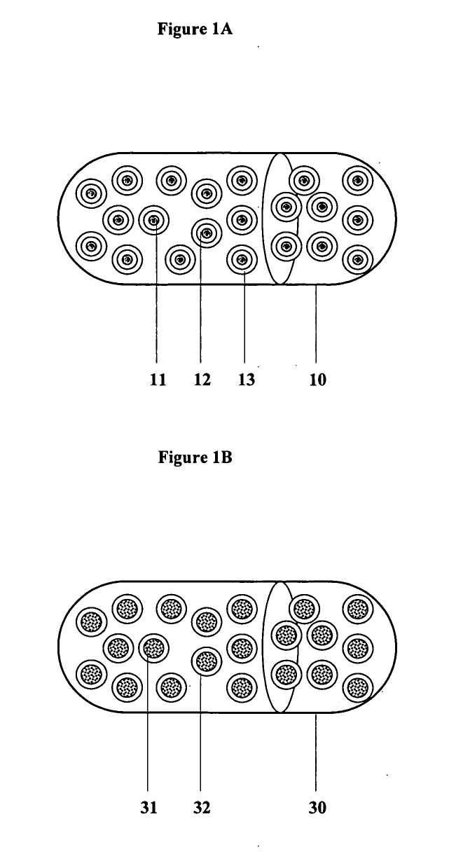

In a preferred embodiment illustrated in FIG. 1A, the solid oral dosage form is a multiparticulate formulation containing drug(s), excipients, and optionally permeation and or dissolution enhancers, encapsulated in a single hard gelatin or cellulose-based capsule, 10, monolithic matrix. The capsule 10 contains multiparticulates 11 of drug(s), excipients, and optionally permeation and or dissolution enhancers. The particulates are optionally coated with one or more layers of release rate controlling polymers or enteric polymers 12 and one layer of a bioadhesive polymer composition 13. The tablet disintegrates quickly in an aqueous medium, releasing its multiparticulate contents.

In another preferred embodiment, illustrated in FIG. 1B, the solid oral dosage form is a multiparticulate formulation, containing drug(s), excipients, a bioadhesive polymer composition, and optionally permeation and or dissolution enhancers, composed in a single hard gelatin or cellulose-based capsule, 30, or monolithic matrix. The capsule contains multiparticulates, 31, of drug(s), excipients, bioadhesive polymer composition, and optionally permeation and or dissolution enhancers. The particulates are optionally coated with one or more layers of release rate controlling polymers or enteric polymers, 32. In another embodiment, the solid oral dosage form is a longitudinally compressed tablet, containing drug, excipients, and dissolution enhancers, composed in a single monolithic layer. The tablet is sealed peripherally with a layer of bioadhesive polymer, leaving the upper and lower sides of the tablet available for drug release.

In another embodiment, the overall shape of the device is designed to be compatible with swallowing. The active agent core can be longitudinally compressed to form a capsule-shaped tablet, which is encapsulated and sealed in a bioadhesive polymeric cylinder. In one embodiment, the core is a multiparticulate containing core, where the active agent is in the form of microparticles. In another embodiment, the active agent core is encapsulated in a bioadhesive polymer cylinder, wherein the tablet is modified to create restricted release openings.

FIG. 1C is a cross-section of a multilayer tablet containing drug in a central matrix of hydrophilic, rate controlling polymers. The inner core is surrounded on two sides by bioadhesive polymer layers, optionally surrounded by an enteric coating. As illustrated in FIG. 1C, the solid oral dosage form is a longitudinally compressed tablet 40 containing one or more drugs, excipients, and optionally permeation and/or dissolution enhancers, disposed in two or more monolithic layers 41 and 42, optionally blocked at one end by a slow-dissolving or non-dissolving passive matrix (also referred to herein as “plug”) 43. The tablet is coated peripherally with a layer of bioadhesive composition 44 leaving the upper side 45 of the tablet available for drug release. First-order and, more advantageously, zero-order release profiles are achievable with this tablet design. The tablet can be designed to provide different immediate release or extended release rates for drugs by changing the composition of the drug layers, or by changing the formulation of the plug. In a preferred embodiment, the solid oral dosage form is a tablet, preferably a trilayer tablet, containing drug in a central matrix of polymer such as hydroxypropylmethylcellulose (“HPMC”) and microcrystalline cellulose (“MCC”) or spray-dried lactose. The inner core is surrounded on two sides by a porous bioadhesive polymer, such as DOPA-BMA polymer or a mixture of bioadhesive p[FA:SA] polymer and Eudragit RS PO. Optionally, the tablet is coated with an enteric coating.

In another embodiment, multiple drug layers are separated by a separating layer and sealed in a bioadhesive polymer cylinder. The resulting capsule can be modified to create restricted release openings. Osmotic systems can be prepared by coating an active agent core with a semi-permeable coating and sealing the coated tablet in a bioadhesive polymer cylinder. In another embodiment, the solid oral dosage form is a longitudinally compressed tablet containing drug, excipients, and dissolution enhancers, composed in two or three monolithic layers, which are separated by slow dissolving passive matrices (also referred to herein as “plugs”). The tablet is coated entirely with a moisture-protective polymer, and then sealed peripherally with a layer of bioadhesive polymer, leaving the upper side, of the tablet available for drug release. The tablet can be designed to provide different immediate release or extended release rates for drugs in a two-pulse or three-pulse fashion by changing the composition or configuration of the drug layers, or by changing the formulation or configuration of the plugs.

In another embodiment, the drug is delivered from an osmotic delivery system. The tablet is coated with a semipermeable membrane. One or both sides of the tablet may be perforated, such as by using a micro-drill or a laser beam to make a micrometer-sized orifice. The tablet is sealed peripherally with a matrix of bioadhesive polymer, leaving the orifice and upper and/or lower sides, of the tablet available for drug release. The semipermeable membrane allows permeation of water into the matrix, leading to the dissolution of drug and creation of osmotic pressure. The increase of osmotic pressure pushes the drug out of the device through the one or more orifice(s) and membrane at controlled rates. Zero-order release profiles are achievable with this tablet design. In another embodiment, the osmotic delivery system is of the “push-pull” design and contains a micronized drug and osmotic agents to draw water across a semi-permeable membrane and a swelling polymer to push the drug out of the device at controlled rates. The entire device is coated with bioadhesive polymers or contains polymer in the matrix of the capsule. The tablet contains an orifice through which the drug is delivered.

In yet another embodiment, a longitudinally compressed tablet containing precompressed inserts of drug and excipients and permeation enhancers and excipients is embedded in a matrix of bioadhesive polymer. Drug is released only at the edge of the tablet and the kinetics of drug release is controlled by the geometry of the inserts.

In the preferred embodiment, the extruded bioadhesive polymer cylinder is prepared via hot-melt extrusion process, where the desired bioadhesive polymer is fed into the extruder as a pellet, flake, powder, etc. along with plasticizer. The materials are blended as they are propelled continuously along a screw through regions of high temperature and pressure to form the polymer extrudate. The extrudate is pushed from the extruder through a die having the desired shape and dimension to form a cylinder. The cylinder is cooled after extrusion. The dimensions of the cylinder can be varied to accommodate the inner core system. The inner diameter of the cylinder can be configured to conform to the desired circumferential dimension of the preformed, pre-pressed inner system containing the therapeutic agent(s). The thickness of the cylinder is determined in part by the polymer/plasticizer type as well its behavior with respect to the external fluid. The bioadhesive nature of the polymer cylinder may also be controlled by mixing different type of polymers and excipients. Inorganic metal oxides may be added to improve the adherence. Pore formers may also be added to control its porosity. Drugs may also be added into the polymer cylinder either as a plasticizer or pore-forming agent. Once formed, the inner system preferably in the form of longitudinally compressed tablet is inserted into the cylinder and two components are fused together to get a finished dosage form.

Extrusion Method for Production of the Hollow Bioadhesive Cylinder

Prior to hot-melt extrusion of the hollow cylinder, the polyanhydride polymer i.e. poly(fumaric-co-sebacic)acid or poly adipic acid and 20% triethyl citrate (based on polymer weight) are mixed in a planetary mixer. Extrusion is performed using either a MP 19 TC25 laboratory scale co-rotating twin screw extruded of APV Baker (Newcastle-under-Lyme, UK) or a Killion extruder (Killian extruder Inc., Cedar Grove, N.J.). Both machines are equipped with a standard screw profile with two mixing sections, an annual die with metal insert for the production of the cylinder and twin screw powder feeder. Typical extrusion conditions are: a screw speed of 5 rpm, a powder feed rate of 0.14 kg/hr and a temperature profile of 125-115-105-80-65° C. from the powder feeder towards the die. The cylinders (internal diameter of 7 mm and wall thickness of 1 mm) are cut into 1 cm long cylinders.

Compression Method for Production of the Hollow Bioadhesive Cylinder

The bioadhesive polymer cylinder may also be formed by a compression process, where the desired bioadhesive polymeric blend is fed into a die of the tabletting machine and compressed using the upper punch attached with a telescopic rod. The telescopic rod pushes the blend and compresses the cylinder. The diameter of the telescopic rod controls the thickness of the bioadhesive cylinder.

Method for Production of the Inner Core System

Inner longitudinally core tablets containing the therapeutic agent and other components are compressed onto a single or multilayer tableting machine equipped with deep fill or regular tooling. For example, the therapeutic agent either alone or in combination with a rate controlling polymer and other excipients is mixed by stirring, ball milling, roll milling or calendaring and pressed into a solid having dimensions conforming to an internal compartment defined by the extruded polymer cylinder. One or more layers containing different therapeutic agents can be included as a multilayer tablet. The inner core system may be a pre-fabricated osmotic system which is inserted into the bioadhesive cylinder with orifices aligned along the open ends of the cylinder.

Method of Insertion of the Inner Core System into the Bioadhesive Cylinder

The preformed inner core with a diameter slightly smaller than the inner diameter of the cylinder is either manually or mechanically inserted into the cylinder and heated to fuse the two units. Alternately, the core insertion into the cylinder may also be done by a positive placement core insertion mechanism on the tableting machine. Initially, the extruded cylinder may be placed into the die of the machine followed by insertion of the compressed core into the internal compartment of the cylinder and the two components compressed to get the finished dosage form. Alternatively, the dosage form is prepared via simultaneous extrusion of the bioadhesive cylinder and expandable inner composition using an extruder capable of such an operation. Alternatively, the dosage form is prepared via compression coating process. The preformed inner core with length similar to the diameter of the die is mechanically inserted over a bed of bioadhesive polymer/excipients blend by a positive core insertion mechanism. After core insertion, additional bioadhesive polymer/excipients blend is added over the core and compressed to get the final dosage form.

The present invention will be further understood by reference to the following non-limiting examples.

### EXAMPLE 1

### Pharmacokinetics of Bioadhesive Gabapentin Tablets (Gabapentin XL) Targeted to the Upper Gastrointestinal tract

Bioadhesive, trilayer tablets, containing about 400 mg gabapentin in the central core layer sandwiched between two bioadhesive layers, were compressed using 0.3287×0.8937″ capsule-shaped dies (Natoli Engineering) at 3000 psi for 3 seconds in a GlobePharma Manual Tablet Compaction Machine (MTCM-1). The composition of the inner core tablet and bioadhesive coating are as follows:

TABLE 3A
Composition of Active Core Layer

| | | mg per | |
|---|---|---|---|
| Component | Function | tablet | % w/w |
| Gabapentin | Active | 397 | 56.1 |
| Hypromellose 4000 cps | Rate-Controlling | 49 | 7.0 |
| | Polymer | | |
| Hypromellose 100 cps | Rate-Controlling | 199 | 28.1 |
| | Polymer | | |
| Emcocel 90M | Filler/binder | 49 | 7.0 |
| Magnesium Stearate | Lubricant | 13 | 1.8 |
| Total | | 707 | 100.0 |

TABLE 3B
Composition of Outer Bioadhesive Layers

| Component | Function | mg per tablet | % w/w |
|---|---|---|---|
| Catechol-grafted | Bioadhesive Polymer | 450 | 90 |
| Butadiene Maleic | | | |
| Anhydride | | | |
| (Spheromer ™ III) | | | |
| Povidone K-30 | Binder | 45 | 9 |
| Magnesium Stearate | Lubricant | 5 | 1 |
| Total | | 500 | 100 |

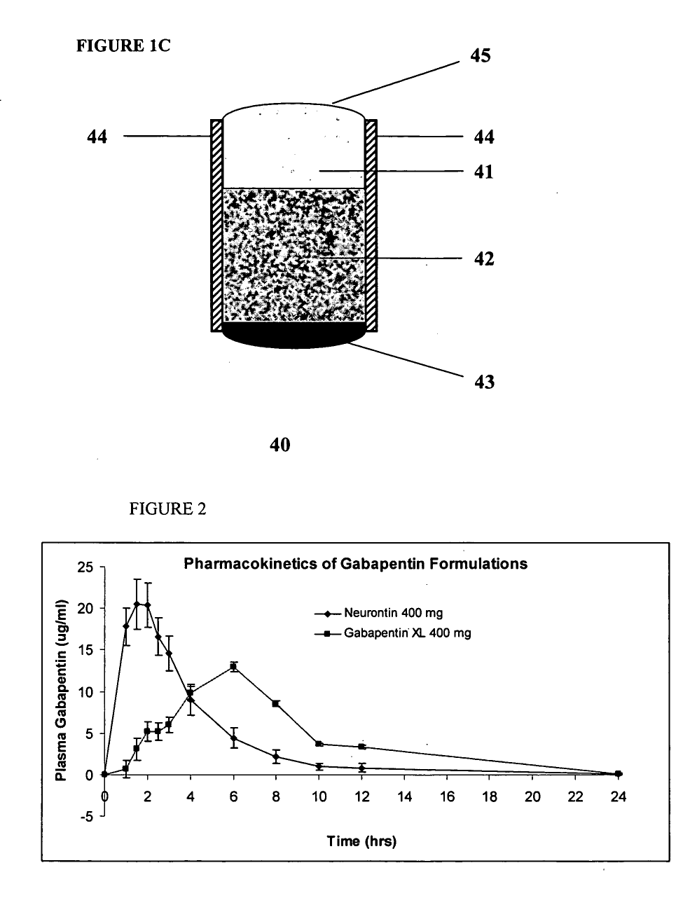

Neurontin immediate release tablets and gabapentin tablets with the composition shown in Tables 3A and 3B were administered to cohorts of six beagle dogs in the fed state and plasma levels of gabapentin were measured using LC/MS/MS, as shown in FIG. 2.

The area under the plasma gabapentin veresus time curve (AUC), maximum concentration (Cₘₐₓ) and time required to achieve Cₘₐₓ (Tₘₐₓ) were calculated and the results are indicated in Table 4 below.

TABLE 4
Area under the Curve, Cmax and Tmax

| | AUC | Cₘₐₓ | Tₘₐₓ |
|---|---|---|---|
| Formulation | ug/ml * hr | ug/ml | hr |
| Neurontin | 88.7 ± 14.0 | 22.9 ± 2.4 | 0.8 ± 0.3 |
| Gabapentin XL | 100.7 ± 11.2 | 16.3 ± 1.9 | 7.0 ± 1.2 |

Gabapentin XL bioadhesive trilayer tablets exceeded the AUC of the immediate release reference form, Neurontin, by more than 10%. Gabapentin is known be absorbed only in the upper small intestine and permeability is limited by carrier-mediated transport in intestinal mucosa. The higher Tmax of the bioadhesive, Gabapentin XL, was comparable to the Neurontin.

### EXAMPLE 2

### Formulation of Sulfasalazine for Colonic Delivery

Targeted delivery of drugs to the colon is considered a useful approach in the treatment of local disorders such as inflammatory bowl diseases (IBD) or systemic absorption of protein/peptide drugs which are degraded in the small intestine. Sulfasalazine is a Biopharmaceutical Classification Class II drug used for treatment of IBD. Sulfasalazine is a prodrug that is enzymatically cleaved by colonic bacterial azoreductase into sulfapyridine, which is nearly completely absorbed by the colon, and to the active pharmaceutical moiety, 5-amino-salicyclic acid (5-ASA), which is minimally absorbed by the colon. 5-ASA is a non steroidal anti-inflammatory drug (NTHE) that acts topically on inflamed colonic mucosa.

Cleavage of sulfasalazine has also been used as an indicator of colonic transit time for solid oral dosage forms. The released sulfapyridine is rapidly absorbed and appearance of sulfapyridine in plasma after dosing has been used to measure colonic transit time and the area under the plasma sulfapyridine vs. time curve has been used as an indicator of topical efficacy of the drug.

Sulfasalazine is available commercially as Azulfidine-EN® tablets containing 500 mg of sulfasalazine coated with the enteric polymer cellulose phthalate, for delayed release in the small intestine. The recommended adult daily dose is 3-5 g administered evenly throughout the day after meals.

Multiparticulate cores were prepared by extrusion spheronization. Sulfasalazine was blended with Emcocel 90M®, Hydroxypropylcellulose EF Pharm, citric acid, sodium lauryl sulfate, Lutrol F127® and Tromethane Base and wet granulated by addition of water while mixing in a Hobart mixer. The granulation was then extruded into rods using a Caleva Model 25 twin-roller extruder and spheronized at 1000 rpm, using a Caleva Model 250 spheronizer, to produce multiparticulate cores. The cores were tray-dried at 50° C. for 18 hrs in a drying oven.

A Spheromer™ III (catechol-grafted anhydride polymer) coating was applied to the sulfasalazine cores by the Wurster-coating method using a Vector MFL.01 laboratory fluidized bed coater. A 10% (w/v) Spheromer™ III solution in methanol/dichloromethane (50:50) containing 0.6% w/v citric acid was sprayed onto the active cores using the following process parameters: inlet temperature=40° C.; atomization pressure=25 psi; coating solution feed rate=6 ml/min; fluidization=250 L/min. The composition of the bioadhesive sulfasalazine multiparticulate formulations is indicated in Table 5 below:

TABLE 5
Composition of Bioadhesive Sulfasalazine Formulation

| | | % w/w | |
|---|---|---|---|
| Component | Function | Batch 1 | Batch 2 |
| Sulfasalazine | Active Pharmaceutical | 53.2 | 53.2 |
| | Ingredient | | |
| Emcocel 90M | Wet Massing Agent | 21.5 | 21.5 |
| Hydroxypropylcellulose EF | Binder | 7.2 | 7.2 |
| Pharm | | | |
| Citric Acid | Acidulant | 4.0 | 4.0 |
| Sodium Lauryl Sulfate | Surfactant/Extrusion Aid | 0.8 | 0.8 |
| Lutrol F127 | Surfactant | 4.0 | 0.8 |
| Tromethane Base | Buffer | NA | 3.2 |
| Spheromer ™ III | Bioadhesive Polymer | 9.3 | 9.3 |
| | Total | 100 | 100 |

1017 mg of multiparticulate beads, containing 500 mg sulfasalazine, were encapsulated in “000” gelatin capsules and manually coated by dipping into a 10% (w/v) solution of Eudragit S100® enteric polymer in acetone containing 10% w/w triethyl citrate as the plasticizer. The dipping procedure was repeated twice with 18 hr intervals of air-drying at 23° C. between coating, to produce a final coating weight gain of approximately 22 mg per capsule.

In vitro Dissolution of Bioadhesive Sulfasalazine Formulations

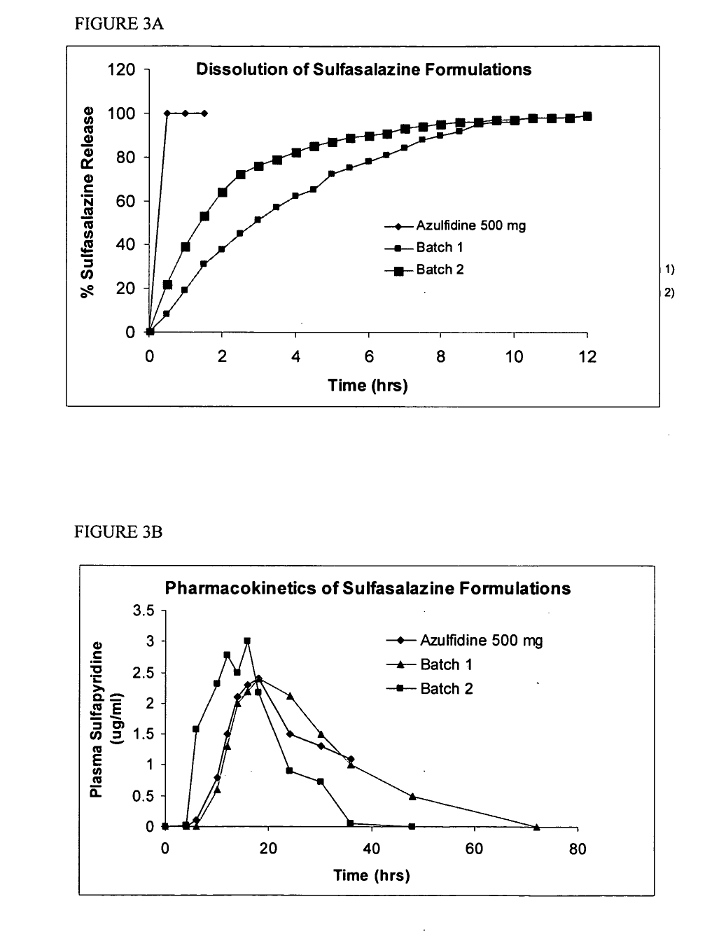

Azulfidine-EN® tablets and bioadhesive polymer coated multiparticulate beads in gelatin capsules (Batch 1 and 2) were tested for dissolution in 900 mL of phosphate buffered saline, pH 7.5 at 37° C. using a USP II apparatus at 100 rpm. The dissolution profiles are shown in FIG. 3A.

Dissolution of Azufidine EN® tablets was complete within 20 min, typical of an immediate release (IR) formulation. The bioadhesive multiparticulate formulations demonstrated controlled release dissolution profiles. Dissolution of Batch 1 multiparticulate beads was 50% complete within 1.5 hrs, 85% complete within 7 hrs and 100% complete by 10 hrs. Dissolution of Batch 2 multiparticulates was 50% complete within 2.5 hrs, 85% complete within 5 hrs and 100% complete by 8 hrs.

The integrity of the Eudragit S100® coated gelatin in acidic medium was tested by observing the capsules for 2 hrs in simulated gastric fluid at 37° C., pH 1.2 in a USP II apparatus at 100 rpm for signs of failure. The capsules did not dissolve during the 2 hrs test period.

In vivo Pharmacokinetic Performance of Sulfasalazine Formulations in Fed Beagle Dogs

The in vivo performance of bioadhesive sulfasalazine formulations was evaluated in beagle dogs. Azulfidine-EN® tablets, 500 mg and Spheromer™ III-coated multiparticulate in capsule formulations (Batch 1 and 2) were administered to cohorts of six beagle dogs in the fed state and plasma levels of sulfapyridine were measured using LC/MS/MS. FIG. 3B shows the plasma concentration profiles of sulfapyridine from different formulations. Table 6 provides the calculated AUC, Cmax and Tmax.

TABLE 6
The area under the plasma sulfapyridine vs. time curve (AUC), maximum
concentration (Cmax) and time required to achieve Cmax (Tmax)

| | AUC | Cₘₐₓ | Tₘₐₓ |
|---|---|---|---|
| Formulation | μg/ml * hr | ug/ml | hr |
| Azulfidine EN Tablets, 500 mg | 44.5 ± 14.0 | 3.6 ± 0.6 | 21.6 ± 3.5 |
| Bioadhesive Formulation (Batch 1) | 47.0 ± 7.6 | 3.6 ± 0.6 | 21.6 ± 3.5 |
| Bioadhesive Formulation (Batch 2 | 57.8 ± 16.0 | 3.4 ± 1.0 | 22.3 ± 3.9 |

Bioadhesive multiparticulate formulations were able to achieve the same or exceeded the performance of the delayed release Azulfidine EN® tablets. The AUC of Batch 2 bioadhesive multiparticulate formulation was 30% higher than that of Azulfidine EN® tablets.

Fluoroscopy Evaluation of Bioadhesive Multiparticulate Beads in Fed Beagle Dog

A fluoroscopy study was conducted in six beagle dogs to ascertain the time and location of multiparticulate beads administered in capsule dosage form. Fed beagle dogs were dosed with capsules containing radio-opaque, barium sulfate-loaded multiparticulate beads coated either with bioadhesive Spheromer™ III polymer or non-bioadhesive cellulose acetate polymer (control). The radio-opaque cores were manufactured by extrusion-spheronization process and coated in the Fluidized bed Wurster coater similar to processes used for the sulfasalazine cores. The bioadhesive beads (1.8 to 2.2 mm in diameter) were encapsulated in “000” gelatin capsules and manually coated with Eudragit S-100® enteric polymer as described above for the sulfasalazine capsules.

Fluoroscopy was performed over the course of 12 hrs. A fluoroscopic image taken at 8 hr post-dosing of a dog fed bioadhesive, Spheromer™ III multiparticulates shows the bioadhesive multiparticulate beads lining the large colon at 8 hrs post dosing. The bioadhesive beads uniformly lined the ascending large bowel, and remained in close apposition to colonic mucosa, without mixing with food. In contrast, non-bioadhesive beads coated with cellulose acetate were uniformly distributed in the lower small bowel, mixed with food, moved freely with peristaltic movements and did not attach to the intestinal mucosa.

### EXAMPLE 3

### Comparison of Sporanox, Spherazole™ IR and Spherazole™ CR Tablets Pharmacokinetics in Dogs

Itraconazole is a synthetic triazole antifungal agent, consisting of a 1:1:1:1 racemic mixture of four diastereomers. It is used for the treatment of fungal infections which are isolated to a small area of the body.

Spherazole™ IR is an immediate release formulation of itraconazole that has lower variability than the innovator product, Sporonox®. The itraconazole is spray-dried with Spheromer™ I bioadhesive polymer to reduce drug particle size and blended with excipients including croscarmellose (superdisintegrant), talc(glidant), microcrystalline cellulose (binder/filler) and magnesium stearate (lubricant). The blend is dry granulated by slugging, to increase bulk density, and subsequently milled, sieved and compressed. The final product is a 900 mg oval tablet containing 100 mg of itraconazole, identical to the Sporonox® dose. The composition of the tablet is 11% itraconazole; 14.8% Spheromer® I; 11.1 % hydroxypropylmethylcellulose (HPMC) 5 cps (E5), 2% Talc, 19.7% Cross-linked carboxymethylcellulose sodium (AcDiSOL), 1% Magnesium Stearate and 40.3% Microcrystalline cellulose (MCC).

Spherazole™ CR is formulated as a trilayer tablet. Itraconazole is dissolved in solvent with Eudragit® E100 and either spray-dried or drug-layered onto MCC cores, blended with HPMC of different viscosities (5,50,100, 4000 cps) and other excipients (corn starch, lactose, microcrystalline cellulose or MCC) to control drug release. The rate controlling inner drug layer is then sandwiched between outer adhesive layers composed of Spheromer™ I or III and optionally Eudragit® RS PO to improve mechanical properties of the bioadhesive layer.

Itraconazole was spray-dried with bioadhesive Spheromer™ co-dissolved in solution to produce 40% Itraconazole w/w loaded particles (Lot 404-109). The spray-dried particles were blended with HPMC 4000 cps and fluid bed granulated using 3% HPMC E5 as the binder. The granulation was filled into “000” gel caps and tested once (n=6/test) in the fed beagle model.

When tested in the “fed” beagle model, the IR formulation has an AUC in the range of 20,000±2000 ng/ml*hr-1, Cmax of 1200± ng/ml, tmax of 2±1 hrs. This performance is equivalent to performance of Sporonox™ in the fed dog model and less variable than the innovator product. Spherazole™ CR when tested in the fed beagle model has an AUC in the range of 20,000±2000 ng/ml*hr-1, Cmax of 600± ng/ml, tmax of 8-20 hrs depending on the particular composition of the rate-controlling core. The performance of the trilayer CR product is similar to Spherazole™ IR and Sporanox® with respect to AUC, however, Cmax is lower by 50%, an important benefit in terms of reduced side effects and drug toxicity. The extended tmax facilitates qd dosing compared to bid dosing for the innovator and IR products. AUC of the gelcap CR formulation was superior to the AUC range for Spherazole™ IR and Sporonox® in the same model.

### EXAMPLE 4

### Human Pharmacokinetics of Bioadhesive Itraconazole Tablets (Spherazole™ CR Type A and B) Versus Sporanox Capsules in Healthy Subjects

Two formulations of bioadhesive, controlled release (CR) trilayer tablets containing 100 mg itraconazole in the central core layer were compressed using 0.3287×0.8937″ capsule-shaped dies (Natoli Engineering) at 3000 psi for 3 seconds in a GlobePharma Manual Tablet Compaction Machine (MTCM-1). The central core formulation was identical for both type A and Type B tablets, however the bioadhesive layer formulations differed. The composition of the tablet is shown in Table 7.

TABLE 7
Composition of Spherazole ™ Tablets

| | | | |
|---|---|---|---|
| | | | % |
| Component | Function | mg per tablet | w/w |

Core Layer

| | | | |
|---|---|---|---|
| 30% | Active Pharmaceutical | 292 | 38.9 |
| Itraconazole/Eudragit | Complex | | |
| E100 Layered onto | | | |
| Microcrystalline | | | |
| Cellulose (Emcocel 90 M) | | | |
| Hypromellose 100 cps | Rate-Controlling | 85 | 11.3 |
| | Polymer | | |
| Hypromellose 5 cps | Rate-Controlling | 255 | 33.9 |
| | Polymer | | |
| Spray-dried | Compressible binder | 116 | 15.5 |
| Lactose(Fast Flo 316) | | | |
| Magnesium Stearate | Lubricant | 2 | 0.3 |
| Total | | 727 | 100.0 |

Bioadhesive Layers (2) Type A

| | | | |
|---|---|---|---|
| Poly (Fumaric-co- | Bioadhesive Polymer | 362 | 73.9 |
| sebacic)anhydride 20:80 | | | |
| polymer | | | |
| (Spheromer ™ I) | | | |
| HydroxypropylCellulose | Binder | 17 | 3.5 |
| Eudragit RS PO | Binder | 108 | 22.1 |
| Magnesium Stearate | Lubricant | 2 | 0.5 |
| Total | | 490 | 100.0 |

Bioadhesive Layers (2) Type B

| | | | |
|---|---|---|---|
| Poly (Fumaric-co- | Bioadhesive Polymer | 292 | 59.5 |
| sebacic)anhydride 20:80 | | | |
| polymer (Spheromer ™ I) | | | |
| Citric Acid | Bioadhesive Excipient | 71 | 14.4 |
| | Acidulant | | |
| Eudragit RS PO | Binder | 108 | 22.1 |
| Hydroxypropyl | Binder | 17 | 3.5 |
| Cellulose | | | |
| Magnesium Stearate | Lubricant | 2 | 0.5 |
| Total | | 490 | 100.0 |

Dissolution testing with Spherazole™ CR tablets (n=6) was performed in 900 ml of simulated gastric fluid (SGF), pH 1.2 in a USP II apparatus at 100 rpm. The results are indicated below.

TABLE 8
Release of Itraconazole from CR Formulations

| Time | Type A | Type B |
|---|---|---|
| hr | % Release | % Release |
| 0 | 0 | 0 |
| 1 | 7.69 | 11.2 |
| 2 | 13.8 | 17.7 |
| 4 | 21.6 | 29.1 |
| 8 | 41.6 | 49 |
| 12 | 76.9 | 80 |
| 16 | 87.1 | 95.9 |
| 24 | 89.9 | 97.4 |

In contrast, dissolution of Sporonox is 85% complete within 60 minutes.

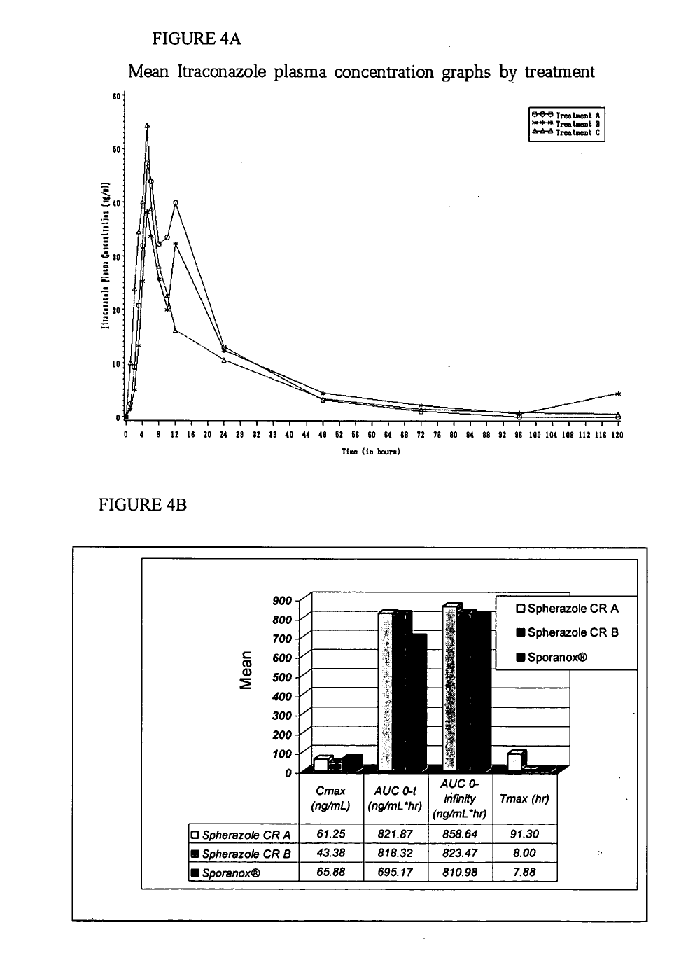

100 mg of itraconazole as Sporanox capsules and Spherics' bioadhesive trilayer tablets (Spherazole™ CR) were administered to 8 volunteers following a light breakfast and plasma levels of itraconazole were measured using LC/MS/MS. The results are shown in FIGS. 4A and 4B. FIG. 4A is a graph of the mean concentration of Itraconazole in plasma in fed volunteers against time following a single 100 mg dose of Treatment A (Spherazole™ CR Type A, Spherics Inc. USA), Treatment B (Spherazole™ CR Type B, Spherics Inc. USA) or Treatment C (Sporanox® 100 mg, Janssen Pharmaceutica Products L.P. USA), n=8. FIG. 4B is a graph of the area under the plasma itraconazole versus time curve (AUC), maximum concentration (Cmax), time to maximum concentration (Tmax) were calculated and are indicated in the figure.

The AUC values for the Spherazole™ CR type A and B formulations were higher than that of Sporanox capsule used as a reference product. For both Type A and B there was an 18% improvement in AUC compared to Sporonox. Cmax was also reduced for the CR products, which is an important advantage because of the Cmax related side-effects associated with Sporanox. Tmax of the CR formulations was elongated compared to Sporanox, which is typical of CR formulations and indicative of gastroretentive behavior.

Trilayer tablets were prepared according to the formulation listed above and tested once (n=6/test) in the fed beagle model. A non-adhesive polymer, Ethocel, was substituted for Spheromer™ I. The AUC of the non-adhesive formulation was similar to the AUC of the adhesive formulation, except that Tmax was reduced from 16 and 19 hrs to 8 hrs in the non-adhesive control and the Cmax was 1049 ng/ml for the non-adhesive control compared to 615 and 691 ng/ml for the adhesive formulation. Incorporating non-adhesive polymer into the outer layers changed the in vivo performance so that it more closely resembled the IR formulation.

The CR versions had considerably reduced variability in Cmax. Both bioadhesive controlled release formulations resulted in lower % CV in Cmax and AUC compared to the reference product. CR type A had a 48% reduction in variability for Cmax and 35% reduction in variability for AUC₀₋ₜ compared to Sporonox. Similarly, CR type B had a 12.2% reduction in variability for Cmax and 27% reduction in variability for AUCO₀₋ₜ compared to Sporanox. Reduction in intersubject variability was as a result of the bioadhesive formulation being less prone to gastric emptying rates. The AUC, Cmax and Tmax of the two experiments were very similar. The AUC of the CR formulation was superior to the AUC range for Spherazole™ IR and Sporonox® in the same model.

### EXAMPLE 5

### In Vitro Dissolution and PK Performance of Acyclovir (Zovirax™) 400 mg Versus CR BioVir Formulations

Zovirax® (Acyclovir) 400 mg, Immediate Release (IR) tablets were compared with tablets prepared with 400 mg acyclovir in a controlled release (CR) formulation, BioVir™, and 400 mg acyclovir, 300 mg in a controlled release formulation and 100 mg in an immediate release formulation (CR+). Trilayer tablets (also referred to herein as “BioVir™ 400 mg”) were prepared using the following formula:

TABLE 9A
Acyclovir Trilayer Tablets
Inner Core: (600 mg)

| | |
|---|---|
| 67.6% | w/w Acyclovir |
| 16.9% | w/w Ethocel 10 Standard FP |
| 11.3% | w/w Glutamic Acid (acidulant) |
| 2.7% | w/w Talc |
| 0.5% | w/w Aerosil 200 |
| 1.0% | w/w Magnesium Stearate |

Outer Layer: (300 mg × 2)

| | |
|---|---|
| 99% | w/w Spheromer III |
| 1% | w/w Magnesium Stearate |

A second trilayer tablet having the composition described above containing 300 mg of acyclovir was produced by direct compression at 3000 psi for 5 seconds. The inner core weighed 444 mg and each outer layer weighed 225 mg.

An immediate release (IR) tablet containing 100 mg of acyclovir was prepared with the following composition and directly compressed at 2000 psi for 1 second.

TABLE 9B
Acyclovir Immediate Release Tablets 600 mg

| | |
|---|---|
| 33% | Zovirax ® granulation |
| 25% | Spray-dried lactose |
| 25% | Microcrystalline cellulose |
| 16.6% | Croscarmellose sodium, NF |
| 0.4% | Magnesium Stearate, NF |

The second trilayer tablet and one tablet of IR formulation were combined (“BioVir 300mg+100 mg IR”).

The trilayer and combined trilayer-IR formulations wered dosed to a fed beagle dog and blood samples were taken different appropriate time intervals.

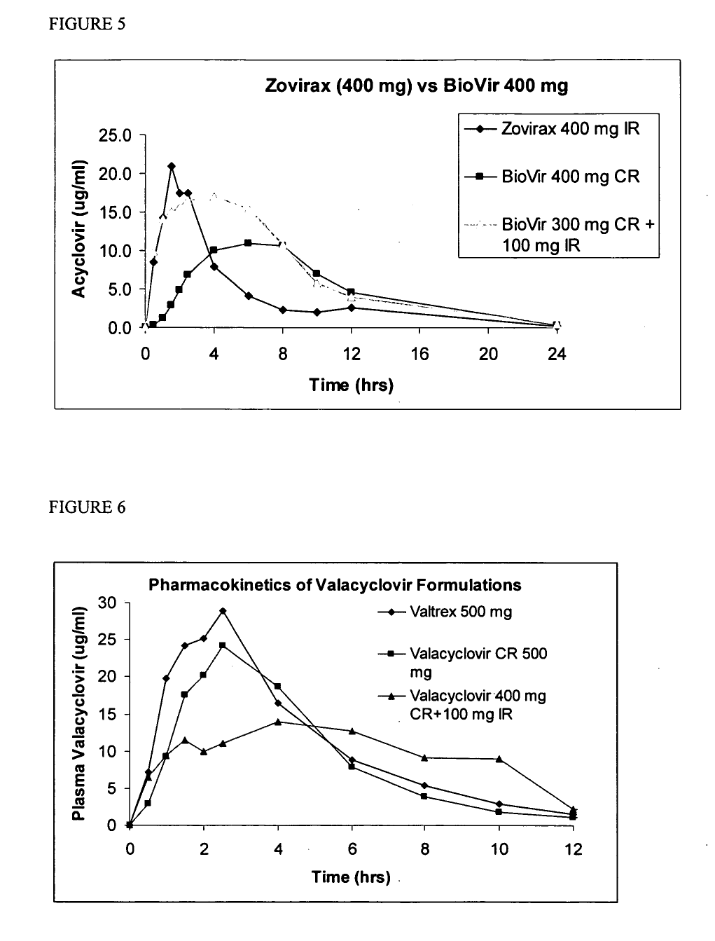

The PK Profiles for Zovirax® (400 mg acyclovir), BioVir™ II (400 mg acyclovir), and BioVir™ II (300 mg acyclovir)+Immediate Release (100 mg acyclovir) (“IR+CR”) are presented in Table 10. The AUC of the IR+CR dosing was 168.2 μg/ml*hr compared to 97.7 μg/ml*hr for Zovirax®, representing a 72% improvement in AUC. Cmax of the IR+CR dosing was 17.0 μg/ml compared to 21 μg/ml for Zovirax®, and Tmax was 4 hrs compared to 1.5 hrs for Zovirax®. The acyclovir concentration in plasma over time is shown in FIG. 5.

TABLE 10
Pharmacokinetic parameters for Zovirax ®
and Controlled Release Formulations

| | Zovirax | BioVir CR | BioVir CR+ |
|---|---|---|---|
| AUC (ug/ml * hr- | 97.7 | 118.7 | 168.2 |
| Cmax (μg/ml) | 21.0 | 10.9 | 17.0 |
| Tmax (hr) | 1.5 | 6.0 | 4.0 |

### EXAMPLE 6

### Comparison of Immediate Release Valacyclovir Tablets (Valtrex®) with Controlled Release Tablets in “Fed” Dog Model

Immediate Release Formulations

Valtrex® is the brand name for valacyclovir, a synthetic nucleoside analogue, manufactured by GlaxoSmithKline for treatment of diseases caused by Herpes virus. Valacyclovir is the prodrug for acyclovir and has greater solubility in water than acyclovir. The bioavailability of valacyclovir is approximately 50% compared to approximately 10-20% for acyclovir.

Controlled Release Formulations

Trilayer tablets described below (referred to as “CR 1” and “CR 2”) were identical in shape (0.3287×0.8937 “00 capsule”) and were compressed at 3000 psi for 5 seconds using the Globe Pharma MTCM machine.

Trilayer tablets were prepared according to the formulation listed below and were tested once (n=6/test) in the fed beagle model and in simulated gastric fluid. The components of the inner core were blended but not granulated. Controlled Release formulation 1 (“CR 1”) was formulated as follows in Table 11:

TABLE 11
Valacyclovir Formulations

| | % w/w |
|---|---|
| Inner Core: (658 mg) | |
| Valacyclovir | 76.2 |
| ETHOCEL ®10 Standard FP | 19.0 |
| (Ethyl cellulose, Dow Chemical Co.) | |
| Talc | 3.0 |
| AEROSIL ® | 0.6 |
| (hydrophilic fumed silica, Degussa AG) | |
| Magnesium Stearate | 1.1 |
| Outer Layer: (300 mg × 2) | |
| Spheromer ™ III | 99.0 |
| Magnesium Stearate | 1.0 |

Controlled Release formulation 2 (“CR 2”) was formulated using the same components in the same proportions as described above for CR 1, except that the inner core contained a total weight of 525 mg. A CR 2 tablet was placed in a hard gelatin capsule (CAPSULGEL®) along with 100 mg of Valacyclovir (VALTREX®, GlaxoSmithKline) to form a solid oral dosage form containing a total of 500 mg valacyclovir/dose (“CR 2 plus IR”).

Test in Fed Beagles

Female beagle dogs were fasted for 24 hrs and chow was returned 30 minutes before dosing (“fed state”) with 1 tablet of VALTREX® (Valacyclovir 500 mg), 1 tablet of CR 1, or 1 capsule containing CR2 plus IR.

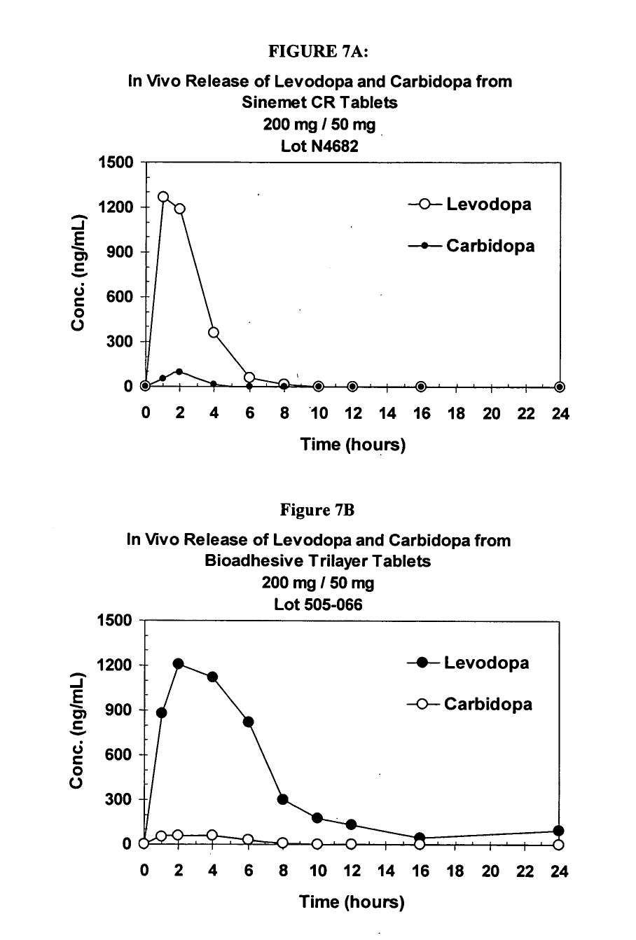

FIG. 6 shows the pharmacokinetic profiles obtained for VALTREX®, CR 1, and CR 2 plus IR. Area under the plasma concentration versus time curve (AUC), maximum plasma concentration (Cmax) and time to maximum plasma concentration (Tmax) were calculated. The AUC, Cmax, and Tmax for each formulation (mean±standard error) is listed in Table 12.

TABLE 12
Pharmacokinetic parameters for Valtrex ® and
Controlled Release Formulations

| | AUC | Cmax | Tmax |
|---|---|---|---|
| Formulation | (μg/ml * hr) | (μg/ml) | (hr) |
| 500 mg Valtrex | 131.7 ± 13.8 | 33.8 ± 6.4 | 2.3 ± 0.5 |
| CR1 | 129.4 ± 15.7 | 26.8 ± 2.2 | 3.8 ± 1.0 |
| CR 2 plus IR | 133.7 ± 24.4 | 21.8 ± 13.9 | 4.3 ± 1.5 |

### EXAMPLE 7

### Sinemet® CR Tablets Containing 200 mg Levodopa and 50 mg Carbidopa (Lot # N4682)

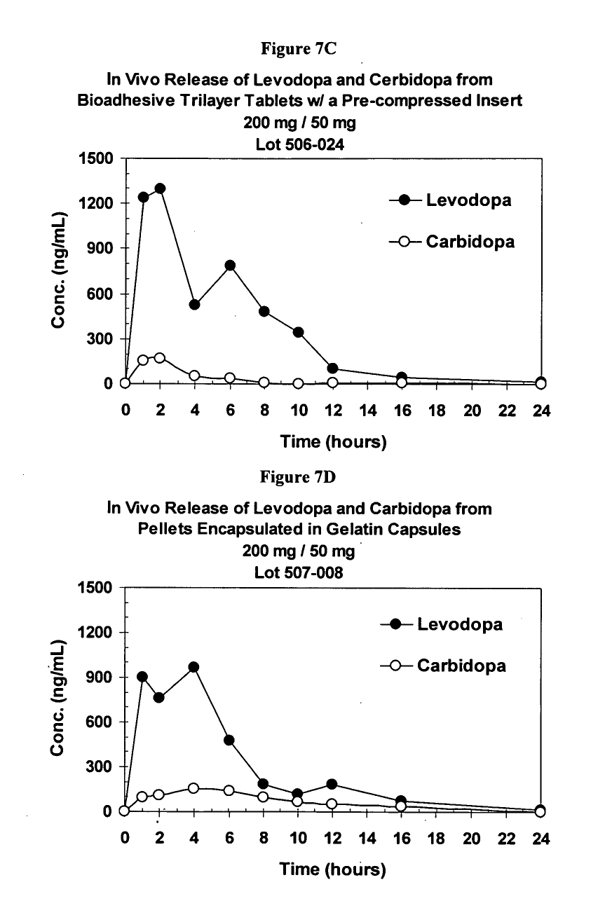

Sinemet® CR tablets were orally administered to beagle dogs that had been fed with ProPlan® Dry Dog Food—Adult, 30 minutes before dosing. The variation of concentration of both Levodopa and Carbidopa in the dogs' plasma is depicted in FIG. 7A. The values of Tₘₐₓ, Cₘₐₓ, and AUC (area under the concentration vs. time curve) were 1 h, 1262.3 ng/mL, and 3903.0 ng.h/mL, respectively.

Bioadhesive trilayer tablets were prepared by sequentially filling a 0.3287″×0.8937″“00 capsule” die (Natoli Engineering) with 250 mg of Spheromer™ III bioadhesive polymer composition, followed by a layer of 466.7 mg of a blend of Levodopa, Carbidopa and pharmaceutically acceptable excipients, followed by an outer layer of 250 mg of Spheromer™ III bioadhesive polymer composition. Trilayer tablets were prepared by direct compression at 3000 psi for 1 second using a GlobePharma Manual Tablet Compaction Machine (MTCM-1). Each tablet contained 200 mg Levodopa and 54 mg Carbidopa monohydrate, equivalent to 50 mg Carbidopa anhydrous. The core composition of tablet is provided in Table 13.

TABLE 13
Levodopa and Carbidopa Formulation

| | Weight (mg) | | |
|---|---|---|---|
| Ingredients | Outer Layer 1 | Core Layer | Outer Layer 2 |
| Levodopa | — | 200.0 | — |
| Carbidopa, Monohydrate | — | 54.0 | — |
| Hypromellose 100 cps | — | 167.2 | — |
| (HPMC) | | | |
| Methocel E5 Prem LV | — | 20.9 | — |
| (HPMC) | | | |
| L-Glutamic Acid HCl | — | 10.4 | — |
| Corn Starch | — | 10.4 | — |
| Spheromer ™ III | 245.0 | — | 245.0 |
| Ethocel 100 Std FP (EC) | 2.5 | — | 2.5 |
| Mg Stearate | 2.5 | 3.8 | 2.5 |
| Total | 250.0 | 466.7 | 250.0 |

The tablets were orally administered to beagle dogs that had been fed with ProPlan® Dry Dog Food—Adult, 30 minutes before dosing. The variation of concentration of both Levodopa and Carbidopa in the dogs' plasma is depicted in FIG. 7B. The values of Tₘₐₓ, Cₘₐₓ, and AUC (area under the concentration vs. time curve) were 2 h, 1210.8 ng/mL, and 8536.7 ng.h/mL, respectively.

Trilayer tablets were prepared by sequentially filling a 0.4375″ ‘round’ die (Natoli Engineering) with 150 mg of Spheromer™ III bioadhesive polymer composition, followed by a 170-mg tablet insert, pre-compressed in a 0.2618″ ‘round’ die (Natoli Engineering) at 500 psi for 1 second using a GlobePharma Manual Tablet Compaction Machine (MTCM-1), followed by a layer of 280 mg of a blend of Levodopa, Carbidopa and pharmaceutically acceptable excipients, followed by an outer layer of 150 mg of a blend of Levodopa, Carbidopa and pharmaceutically acceptable excipients. Trilayer tablets were prepared by direct compression at 3000 psi for 1 second using the GlobePharma tablet press. Each tablet contained 200 mg Levodopa and 54 mg Carbidopa monohydrate, equivalent to 50 mg Carbidopa anhydrous. The core composition of tablet is provided in Table 14.

TABLE 14
Levodopa and Carbidopa Formulation

| | Weight (mg) | | | |
|---|---|---|---|---|
| | Outer | | Core | Outer |
| Ingredients | Layer 1 | Insert | Layer | Layer 2 |
| Levodopa | 40.00 | 40.00 | 120.0 | — |
| Carbidopa, Monohydrate | 10.80 | 10.80 | 32.4 | — |
| Hypromellose 100 cps (HPMC) | — | — | 106.5 | — |
| Methocel E5 Prem LV (HPMC) | — | — | 6.4 | — |
| L-Glutamic Acid HCl | — | — | 6.2 | — |
| Corn Starch | — | — | 6.2 | — |
| Ludipress | 98.45 | 118.45 | — | — |
| Spheromer ® III | — | — | — | 98.0 |
| Ethocel 100 Std FP (EC) | — | — | — | 1.5 |
| Mg Stearate | 0.75 | 0.75 | 2.3 | 0.5 |
| Total | 150.00 | 170.00 | 280.0 | 100.0 |

The tablets were orally administered to beagle dogs that had been fed with ProPlan® Dry Dog Food—Adult, 30 minutes before dosing. The variation of concentration of both Levodopa and Carbidopa in the dogs' plasma is depicted in FIG. 7C. The values of Tₘₐₓ, Cₘₐₓ, and AUC (area under the concentration vs. time curve) were 2 h, 1297.1 ng/mL, and 8104.1 ng.h/mL, respectively.

Two formulations of Levodopa-Carbidopa pellets were prepared by low shear granulation followed by extrusion-spheronization. One formulation, hereinafter referred to as ‘component 1’, was used without coating. Hereinafter referred to as ‘component 2’, pellets were coated in a Wurster fluid bed system with a first layer of Eudragit® RL 100, 2.2 grams per 100 grams core particles, and with a second outer layer of Spheromer™ III polymer, 4 grams per 100 grams core particles. Gelatin capsules, size 00, were filled with 91 mg of component 1 and 386 mg of component 2. Each capsule contained 200 mg Levodopa and 50 mg Carbidopa (anhydrous). The dry composition of core pellets is provided in Table 15.

TABLE 15
Levodopa and Carbidopa Formulation

| | Weight (%) | |
|---|---|---|
| Ingredients | Component 1 | Component 2 |
| Levodopa | 44.0 | 44.0 |
| Carbidopa, Monohydrate | 11.9 | 11.9 |
| Emocel 90M (Microcrystalline | 26.0 | 27.1 |
| cellulose) | | |
| Ac-Di-Sol (Croscarmellose | 6.1 | — |
| Sodium) | | |
| Klucel EF Pharm (HPC) | 5.0 | 10.0 |
| Fast-Flo no. 316 (Lactose, | 5.0 | 5.0 |
| Monohydrate) | | |
| Citric acid | 1.0 | 1.0 |
| Sodium Lauryl Sulfate | 1.0 | 1.0 |
| Total | 100.0 | 100.0 |

The capsules were orally administered to beagle dogs that had been fed with ProPlan® Dry Dog Food—Adult, 30 minutes before dosing. The variation of concentration of both Levodopa and Carbidopa in the dogs' plasma is depicted in FIG. 7D.

The values of Tₘₐₓ, Cₘₐₓ, and AUC (area under the concentration versus time curve) were 4 h, 966.3 ng/mL, and 6558.2 ng.h/mL, respectively.

The results demonstrate that the controlled release, bioadhesive formulations had substantially higher AUC as compared to control Sinemet® CR.

Modifications and variations of the present invention will be obvious to those skilled in the art from the foregoing detailed description and are intended to come within the scope of the appended claims.

We claim:

1. An oral formulation for administration to the plasma of an individual via a site in the gastrointestinal tract comprising
a therapeutic, prophylactic or diagnostic active ingredient to be delivered,
one or more controlled release element, and
one or more bioadhesive element,
wherein the formulation releases the active ingredient into the plasma in coordination with retention of the active ingredient at a site in the gastrointestinal tract, resulting in a greater efficacy as measured by area under the curve for the plasma concentration over time than a reference formulation consisting of the active ingredient in the absence of the controlled release and/or bioadhesive elements.

2. An oral formulation for administration to the plasma of an individual via a site in the gastrointestinal tract comprising
a therapeutic, prophylactic or diagnostic active ingredient to be delivered,
one or more controlled release element, and
one or more bioadhesive element,
wherein the formulation releases the active ingredient into the plasma in coordination with retention of the active ingredient at a site in the gastrointestinal tract, wherein the bioadhesive is exposed at the surface of the formulation after a period of time substantially the same as the time that the drug arrives at the site in the gastrointestinal tract.

3. The oral formulation of claim 1 comprising an active ingredient in a bioadhesive polymeric matrix or bioadhesive coated matrix, wherein the bioadhesive polymer comprises a water insoluble hydrophobic backbone and mucophilic functional groups.

4. The oral formulation of claim 1 wherein the bioadhesive polymer is a water-insoluble hydrophobic polymer selected from the group consisting of polyanhydrides, poly(meth)acrylate, polyhydroxy acids, polyesters, and copolymers thereof.

5. The oral formulation of claim 1 wherein the active ingredient is in the form of microspheres releasing at least 40% of the active ingredient into the gastrointestinal tract, or into water, in less than about 30 minutes.

6. The oral formulation of claim 1 comprising an enteric coating that dissolves in specific regions of the gastrointestinal tract to release active ingredient and to expose the bioadhesive element.

7. The oral formulation of claim 1 remaining substantially intact during the course of drug release and gastrointestinal transit.

8. The oral formulation of claim 1 hydrating (less then 10%) and disintegrating as it moves through the gastrointestinal tract.

9. The oral formulation of claim 1 comprising pharmaceutical acceptable polymers to increase mechanical strength and/or elasticity of the formulation.

10. The oral formulation of claim 1 comprising inorganic or organic pore formers to increase the porosity and permeability of the formulation.

11. The oral formulation of claim 1 comprising enteric-soluble polymers to aid in disintegration of the oral formulation.

12. The oral formulation of claim 1 comprising wicking agents to control hydration and modulate the bioadhesion behavior of the formulation.

13. The oral formulation of claim 1 wherein the active ingredient is a BCS class I drug.

14. The oral formulation of claim 13 wherein the active ingredient is selected from the group consisting of gabapentin, valacyclovir HCl, levodopa, carbidopa, metformin, and ranitidine HCl.

15. The oral formulation of claim 1 wherein the active ingredient is a BCS class II active ingredient.

16. The oral formulation of claim 15 wherein the active ingredient is selected from the group consisting of itraconazole, acyclovir, and sulfasalazine

17. The oral formulation of claim 1 wherein the active ingredient is a BCS class III active ingredient.

18. The oral formulation of claim 17 wherein the active ingredient is selected from the group consisting of abacavir sulfate, amiloride HCl, atropine sulfate, chloramphenicol, folic acid, hydrochlorthazide, lamivudine, methyldopa, mefloquine HCl, penicillamine, pyrazinamide, salbutamol sulfate, valproic acid, stavudine, ethosuximide, ergometrine maleate, colchicines, didanosine, cimetidine, ciprofloxacin, neomycin B, captopril, Atenolol, and Caspofingin.

19. The oral formulation of claim 1 wherein the active ingredient is a BCS class IV active ingredient.

20. The oral formulation of claim 19 wherein the active ingredient is selected from the group consisting of acetazolamide, allopurinol, dapsone, doxycycline, paracetamol, nalidixic acid, clorothiazide, tobramycin, cyclosporin, tacrolimus, and paclitaxel.

21. The oral formulation of claim 1 wherein the formulation is a solid oral dosage formulation selected from the group consisting of tablets, capsules, minitabs, filled tablets, and osmotic tablets.

22. The oral formulation of claim 1, wherein the active ingredient is in the form of particles or granules.

23. The oral formulation of claim 1 further comprising a permeation or absorption enhancer.

24. The oral formulation of claim 23 wherein the enhancer is selected from the group consisting of sodium caprate, ethylenediamine tetra(acetic acid) (EDTA), citric acid, lauroylcarnitine, palmitoylcarnitine, tartaric acid, Vitamin E, TPGS and other agents that increase gastrointestinal permeability.

25. The oral formulation of claim 1 in the form of a trilayer tablet.

26. The oral formulation of claim 1 in the form of a longitudinally compressed tablet.

27. The oral formulation of claim 26 wherein the tablet is composed of a single monolithic layer.

28. The oral formulation of claim 26 wherein the tablet is composed of multiple monolithic layers.

29. The oral formulation of claim 22 wherein the particles or granules are coated with bioadhesive and encapsulated in a hard gelatin or polysaccharide capsule.

30. The oral formulation of claim 22 wherein the particles or granules are coated with bioadhesive and encapsulated in a rapidly disintegrating tablet.

31. The oral formulation of claim 1 further comprising an immediate release formulation.

32. The oral formulation of claim 1 comprising an enteric coating polymer dissolving a pH between 1 and 3 for release of active ingredient in the stomach.

33. The oral formulation of claim 1 comprising a coating polymer dissolving a pH between 4 and 6 for release of active ingredient in the small intestine.

34. The oral formulation of claim 1 comprising a coating polymer dissolving a pH between 7 and 8 for release of active ingredient in the colon.

35. The oral formulation of claim 1 wherein the bioadhesive comprises a polymer backbone substituted with one or more catechols.

36. The oral formulation of claim 35, wherein the catechol is 3,4 -dihydroxyphenylalanine (DOPA).

37. The oral formulation of claim 35, wherein the polymeric backbone is a hydrophobic polymer.

38. The oral formulation of claim 37, wherein the hydrophobic polymer is selected from the group consisting of polyanhydrides, polyacrylates, polyorthoesters, polyesters, and polyhydroxy acids.

39. The oral formulation of claim 1 wherein the bioadhesive comprises a polymer comprising DOPA bound thereto.

40. The oral formulation of claim 1 wherein the bioadhesive comprises anhydride oligomers.

41. The oral formulation of claim 1 wherein the bioadhesive comprises a metal oxide.

42. The oral formulation of claim 1 wherein the active ingredient is released into the buccal/sublingual area of the gastrointestinal tract.

43. The oral formulation of claim 1 wherein the active ingredient is released into the stomach.

44. The oral formulation of claim 43 for treatment or prevention of ulcers or the symptoms of ulcers comprising at least one antibiotic and one or more agents selected from the group consisting of antibiotics, H₂ blockers, proton pump inhibitors, and stomach-lining protectors.

45. The formulation of claim 44 wherein the agents are selected from the group consisting of amoxicillin, tetracycline, metronidazole, clarithromycin, cimetidine, ranitidine, famotidine, nizatidine, omeprazole, lansoprazole, rabeprazole, esomeprazole, pantoprozole, and bismuth subsalicylate.

46. The oral formulation of claim 1 wherein the active ingredient is released into the colon.

47. The oral formulation of claim 2 wherein Cmax is different than a reference formulation consisting of the active ingredient in the absence of the controlled release and/or bioadhesive elements.

48. The oral formulation of claim 47 having greater efficacy of uptake as measured by area under the curve for the plasma concentration over time than a reference formulation consisting of the active ingredient in the absence of the controlled release and/or bioadhesive elements.
